(19) United States
(12) Patent Application Publication
(10) Pub. No.: US 2023/0080534 A1
(43) Pub. Date: Mar. 16, 2023

(54) SEMG2 ANTIBODY AND USE THEREOF

(71) Applicant: SHANGHAI BIOTROY BIOTECHNIQUE CO., LTD., Beijing (CN)

(72) Inventor: Zhaoli LI, Beijing (CN)

(21) Appl. No.: 17/794,598

(22) PCT Filed: Jan. 21, 2021
(86) PCT No.: PCT/CN2021/073100

§ 371 Date: Jul. 21, 2022

(30) Foreign Application Priority Data
Jan. 21, 2020 (CN) 202010072952.X

(51) Int. Cl. C07K 16/18; C07K 14/47; A61K 47/68; A61K 45/06; A61K 39/395; A61K 38/17; A61P 35/00

(52) U.S. Cl. CPC C07K 16/18; C07K 14/47; A61K 47/6843; A61K 45/06; A61K 39/3955; A61K 38/1709; A61P 35/00; A61K 2039/505

(57) ABSTRACT

The present invention provides a compound agonizing or antagonizing the interaction between SEMG2 and CD27, comprising a small molecule inhibitor, a polypeptide, an antibody, or an antigen-binding fragment. The present invention further discloses methods of preparing antibodies for blocking the binding between SEMG2 and CD27 using the polypeptides as an immunogen with high efficiency. The present invention discloses methods of promoting anti- tumor immunity by blocking the contact of SEMG2 expressed by tumor cells with CD27 expressed by immune cells, also discloses a screening method for screening a therapeutic drug by blocking the binding between SEMG2 and CD27.

### TECHNICAL FIELD

The invention relates to the field of biomedicine, specifically to a SEN1G2 antigenic epitope peptide and the use thereof.

### BACKGROUND OF THE INVENTION

The CD27 molecule belongs to the tumor necrosis factor receptor (TNFR) superfamily and is a type I membrane protein with a molecular weight of about 55 kDa, and exists as a dimer of two monomers linked by a disulfide bond. CD27 is mainly expressed in lymphocytes. Recent studies based on CD27 knockout mice have shown that activation of the CD27 signaling pathway can increase the infiltration of suppressor T cells (Treg) in solid tumors and reduce anti-tumor immunity (Claus C. Riether C, Schürch C, Matter M S, Hilmenyuk T, Ochsenbein A F. Cancer Res. 2012 Jul. 15; 72 (14):3664-76). Consistently, the study also found that Treg cells in skin tissue fail to perform normal immune regulation functions after losing CD27 expression (Remedios K A, Zirak B. Sandoval P M, Lowe M M, Boda D, Henley E et al., Sci Immunol. 2018. Dec. 21; 3(30).pii:eaau2042). Furthermore, activation of CD27 increases Treg numbers and reduces atherosclerosis in hyperlipidemic mice (Winkels H, Meiler S, Lievens D, Engel D, Spitz C, Bürger C. et al., Eur Heart J 2017; 38(48):3590-3599). The recent studies consistently demonstrate that CD27 plays an important role in the functional activation of specific Treg cells (including tumor-infiltrating Treg), and therefore avoiding the activation of CD27 expressed by tumor-infiltrating Treg cells is a potential cancer treatment strategy.

Binding to ligands activates the downstream signal transduction of CD27, and the currently known CD27 ligand molecule is CD70. CD70 is a 193 amino acid polypeptide with a hydrophilic N-terminal domain of 20 amino acids and a C-terminal domain containing 2 potential N-linked glycosylation sites, belonging to the TNF family (Goodwin, R. G. et al. (1993) Cell 73:447-56; Bowman et al. (1994) Immunol 152:1756-61). These properties suggest that CD70 is a type II transmembrane protein with an extracellular C-terminal portion. CD70 is transiently present on activated T and B lymphocytes and dendritic cells (Hintzen et al., (1994) J. Immunol. 152:1762-1773; Oshima et al., (1998) Int. Immunol. 10:517-26; Tesselaar et al., (2003) J. Immunol. 170:33-40). In addition to normal cells, CD70 expression has been reported in different types of cancer, including renal cell carcinoma, metastatic breast cancer, brain tumor, leukemia, lymphoma, and nasopharyngeal carcinoma (Junker et al., J. Urol. 2005; 173: 2150-3; Sloan et al., Am J Pathol. 2004; 164:315-23; Held-Feindt and Mentlein et al., Int J Cancer 2002; 98:352-6). Currently, blocking the binding between CD70 and CD27 is a strategy being investigated for tumor immunotherapy.

Previous studies have not suggested that CD27 has other ligands than CD70. However, novel ligands of immune checkpoint pathway receptors (especially novel ligands expressed by tumor cells with relatively high specificity) are of great significance for the development of more effective anti-tumor treatments. The present invention aims to develop new anti-tumor treatments and drugs.

### SUMMARY OF THE INVENTION

In one aspect, the invention discloses a compound agonizing or antagonizing an interaction between SEMG2 and CD27. Wherein, the interaction between SEMG2 and CD27 is located on the amino acid site at positions 497, 498, 499, 500, 501 502, 503, 504, 505, 506, and 508 of SEMG2, and the amino acid sequence of the SEMG2 protein is shown in SEQ ID NO:1. Wherein, the compound is a small molecule inhibitor, polypeptide, antibody, or antigen-binding fragment.

In one embodiment, the invention discloses a polypeptide, the polypeptide comprises an amino acid sequence of SEQ ID NO:2 (QIEKLVEGKS), SEQ ID NO:86 (QIEKLVEGKS(x)I(x)), SEQ ID NO:87 (QIEKLVEGKS(x)I), or SEQ ID NO:88 (QIEKLVEGKS(x)); preferably the polypeptide comprises an amino acid sequence of SEQ ID NO:3 (QIEKLVEGKSQIQ), SEQ ID NO:4 (QIEKLVEGKSQ), or SEQ ID NO:5 (QIEKLVEGKSQI), or an amino acid sequence at least 90% identity to an amino acid sequence as provided in SEQ ID NOs: 2-5. Wherein, the polypeptide agonizes the interaction between SEMG2 and CD27. Wherein, the amino acid site of the interaction between SEMG2 and CD27 is located at positions 497, 498, 499, 500, 501, 502, 503, 504, 505, 506 and 508 of SEMG2, and the amino acid sequence of the SEMG2 protein is shown in SEQ ID NO:1.

In one embodiment, the invention discloses an antibody specifically binding to native or mutant SEMG2 protein, the antibody binds to an antigenic epitope peptide derived from SEMG2 protein, and the antigenic epitope peptide comprises an amino acid sequence of SEQ ID NO:2 (QIEKLVEGKS), SEQ ID NO:3 (QIEKLVEGKSQIQ), SEQ ID NO:4 (QIEKLVEGKSQ), or SEQ ID NO:5 (QIEKLVEGKSQI). Wherein, the antibody antagonizes the interaction between SEMG2 and CD27.

In one embodiment, the invention discloses an antibody specifically binding to native or mutant SEMG2 protein, the antibody recognizes at least one amino acid residue at positions 497, 498, 499, 500, 501, 502, 503, 504, 505, 506 and 508 of the native SEMG2 protein or recognizes an amino acid residue in the corresponding position of the mutant SEMG2 protein, the amino acid sequence of the native SEMG2 protein is shown in SEQ ID NO:1. Wherein, the antibody antagonizes the interaction between SEMG2 and CD27.

In one embodiment, the invention discloses an antibody specifically binding native or mutant SEMG2 protein, wherein the antibody comprises a heavy chain variable region and a light chain variable region, the heavy chain variable region comprises HCDR1, HCDR2 and HCDR3 defined by IMGT; and the light chain variable region comprises LCDR1, LCDR2 and LCDR3 defined by IMGT,

the HCDR1 consists of or comprises an amino acid sequence selected from the group consisting of SEQ ID NOs:6-11, SEQ ID NOs:60-61 and SEQ ID NO:76;

the HCDR2 consists of or comprises an amino acid sequence selected from the group consisting of SEQ ID NOs:12-16 and SEQ ID NOs:62-64;

the HCDR3 consists of or comprises an amino acid sequence selected from the group consisting of SEQ ID NOs:17-20, SEQ ID NOs:65-67 and SEQ ID NOs:77-81;

the LCDR1 consists of or comprises an amino acid sequence selected from the group consisting of SEQ ID NOs:21-25, SEQ ID NOs:68-70 and SEQ ID NO:82;

the LCDR2 consists of or comprises an amino acid sequence selected from the group consisting of SEQ ID NOs:26-29, SEQ ID NOs:71-72, SEQ ID NOs:83-84 and SEQ NO:28;

the LCDR3 consists of or comprises an amino acid sequence selected from the group consisting of SEQ ID NOs:30-34, SEQ ID NOs:73-75, SEQ ID NO:85 and SEQ ID NO:99.

In one specific embodiment, the CDR sequence of the antibody is selected from any one of the combinations in (a)-(k):
(a) the HCDR1 comprises the amino acid sequence of SEQ ID NO:6; the HCDR2 comprises the amino acid sequence of SEQ ID NO:12; the HCDR3 comprises the amino acid sequence of SEQ ID NO:17; the LCDR1 comprises the amino acid sequence of SEQ ID NO:21; the LCDR2 comprises the amino acid sequence of SEQ ID NO:26; the LCDR3 comprises the amino acid sequence of SEQ ID NO:30;
(b) the HCDR1 comprises the amino acid sequence of SEQ ID NO:7; the HCDR2 comprises the amino acid sequence of SEQ ID NO:13; the HCDR3 comprises the amino acid sequence of SEQ ID NO:18; the LCDR1 comprises the amino acid sequence of SEQ ID NO:22; the amino acid sequence of LCDR2 comprises the amino acid sequence of SEQ ID NO:27; the LCDR3 comprises the amino acid sequence of SEQ ID NO: 31 or SEQ ID NO:99;
(c) the HCDR1 comprises the amino acid sequence of SEQ ID NO:6; the HCDR2 comprises the amino acid sequence of SEQ ID NO:16; the HCDR3 comprises the amino acid sequence of SEQ ID NO:17; the LCDR1 comprises the amino acid sequence of SEQ ID NO:21; the LCDR2 comprises the amino acid sequence of SEQ ID NO:26; the LCDR3 comprises the amino acid sequence of SEQ ID NO:30;
(d) the HCDR1 comprises the amino acid sequence of SEQ ID NO:8; the HCDR2 comprises the amino acid sequence of SEQ ID NO:13; the HCDR3 comprises the amino acid sequence of SEQ ID NO:18; the LCDR1 comprises the amino acid sequence of SEQ ID NO:23; the LCDR2 comprises the amino acid sequence of SEQ ID NO:27; the LCDR3 comprises the amino acid sequence of SEQ ID NO:32;
(e) the HCDR1 comprises the amino acid sequence of SEQ ID NO:9; the HCDR2 comprises the amino acid sequence of SEQ ID NO:14; the HCDR3 comprises the amino acid sequence of SEQ ID NO:19; the LCDR1 comprises the amino acid sequence of SEQ ID NO:24; the LCDR2 comprises the amino acid sequence of SEQ ID NO:28; the LCDR3 comprises the amino acid sequence of SEQ TD NO:33;
(f) the HCDR1 comprises the amino acid sequence of SEQ ID NO:10; the HCDR2 comprises the amino acid sequence of SEQ II3 NO:15; the HCDR3 comprises the amino acid sequence of SEQ ID NO:20; the LCDR1 comprises the amino acid sequence of SEQ ID NO:25; the LCDR2 comprises the amino acid sequence of SEQ ID NO:29; the LCDR3 comprises the amino acid sequence of SEQ ID NO:34;
(g) the HCDR1 comprises the amino acid sequence of SEQ ID NO:11; the HCDR2 comprises the amino acid sequence of SEQ II3 NO:15; the HCDR3 comprises the amino acid sequence of SEQ ID NO:20; the LCDR1 comprises the amino acid sequence of SEQ ID NO:25; the LCDR2 comprises the amino acid sequence of SEQ ID NO:29; the LCDR3 comprises the amino acid sequence of SEQ ID NO:34;
(h) the HCDR1 comprises the amino acid sequence of SEQ ID NO:60; the HCDR2 comprises the amino acid sequence of SEQ NO:62; the HCDR3 comprises the amino acid sequence of SEQ ID NO:65; the LCDR1 comprises the amino acid sequence of SEQ ID NO:68; the LCDR2 comprises the amino acid sequence of SEQ ID NO:71; the LCDR3 comprises the amino acid sequence of SEQ ID NO:7:3;
(i) the HCDR1 comprises the amino acid sequence of SEQ ID NO:61; the HCDR2 comprises the amino acid sequence of SEQ ID NO:63; the HCDR3 comprises the amino acid sequence of SEQ ID NO:66; the LCDR1 comprises the amino acid sequence of SEQ ID NO:69; the LCDR2 comprises the amino acid sequence of SEQ ID NO:72; the LCDR3 comprises the amino acid sequence of SEQ ID NO:74;
(j) the HCDR1 comprises the amino acid sequence of SEQ NO:60; the HCDR2 comprises the amino acid sequence of SEQ ID NO:64; the HCDR3 comprises the amino acid sequence of SEQ ID NO:67; the LCDR1 comprises the amino acid sequence of SEQ ID NO:70; the LCDR2 comprises the amino acid sequence of SEQ ID NO:28; the LCDR3 comprises the amino acid sequence of SEQ ID NO:75;
(k) the HCDR1 comprises the amino acid sequence of SEQ ID NO:60 or 76; the HCDR2 comprises the amino acid sequence of SEQ ID NO:64 or 62; the HCDR3 comprises the amino acid sequence of SEQ ID NO:77, 78 or 79; and/or

the LCDR1 comprises the amino acid sequence of SEQ ID NO:70 or 82; the LCDR2 comprises the amino acid sequence of SEQ ID NO:28, 83 or 84; the LCDR3 comprises the ammo acid sequence of SEQ ID NO:75 or 85.

In one embodiment, the invention discloses an antibody binding to native or mutant SEMG2 protein specifically, wherein the antibody comprises a heavy chain variable region and a light chain variable region,

the heavy chain variable region comprises an amino acid sequence selected from the group consisting of SEQ ID NOs:35-41, 48-51, 54-56 and 96-100, or an amino acid sequence at least 70%, 80%, 90%, 95% or 99% identity to any sequences of SEQ ID NOs:35-41, 48-51, 54-56 and 96-100;

the light chain variable region comprises the amino acid sequence selected from the group consisting of SEQ ID NOs:4247, 52-53, 57-69 and 101-103, or an amino acid sequence at least 70%, 80%, 90%, 95% or 99% identity to any sequences of SEQ ID NOs:42-47, 52-53, 57-69 and 101-103.

In one specific embodiment, the heavy chain variable region and the light chain variable region are selected from any one of the combinations in (a)-(o):
(a) the heavy chain variable region comprises SEQ ID NO:35 or an amino acid sequence at least 70%, 80%, 90%, 95% or 99% identity to SEQ ID NO:35; the light chain variable region comprises SEQ ID NO:42 or an amino acid sequence at least 70%, 80%, 90%, 95% or 99% identity to SEQ ID NO:42;
(b) the heavy chain variable region comprises SEQ ID NO:36 or an amino acid sequence at least 70%, 80%, 90%, 95% or 99% identity to SEQ ID NO:36; the light chain variable region comprises SEQ ID NO:43 or an amino acid sequence at least 70%, 80%. 90%, 95% or 99% identity to SEQ ID NO:43;
(c) the heavy chain variable region comprises SEQ ID NO:37 or an amino acid sequence at least 70%, 80%, 90%, 95% or 99% identity to SEQ ID NO:37; the light chain variable region comprises SEQ ID NO:44 or an amino acid sequence at least 70%, 80%. 90%, 95% or 99% identity to SEQ IIS NO:44;
(d) the heavy chain variable region comprises SEQ ID NO:38 or an amino acid sequence at least 70%, 80%, 90%, 95% or 99% identity to SEQ ID NO:38; the light chain variable region comprises SEQ ID NO:45 or an amino acid sequence at least 70%, 80%. 90%, 95% or 99% identity to SEQ IIS NO:45;
(e) the heavy chain variable region comprises SEQ ID NO:39 or an amino acid sequence at least 70%, 80%, 90%, 95% or 99% identity to SEQ ID NO:39; the light chain variable region comprises SEQ ID NO:46 or an amino acid sequence at least 70%, 80%, 90%, 95% or 99% identity to SEQ ID NO:46;
(f) the heavy chain variable region comprises SEQ ID NO:40 or an amino acid sequence at least 70%, 80%, 90%, 95% or 99% identity to SEQ ID NO:40; the light chain variable region comprises SEQ ID NO:47 or an amino acid sequence at least 70%, 80%, 90%, 95% or 99% identity to SEQ ID NO:47;
(g) the heavy chain variable region comprises SEQ ID NO:41 or an amino acid sequence at least 70%, 80%, 90%, 95% or 99% identity to SEQ ID NO:41; the light chain variable region comprises SEQ ID NO:47 or an amino acid sequence at least 70%, 80%, 90%, 95% or 99% identity to SEQ ID NO:47;
(h) the heavy chain variable region comprises SEQ ID NO:48, 49, 50, 51 or amino acid sequence at least 70%, 80%, 90%, 95% or 99% identity to SEQ ID NO:48, 49, 50 or 51; the light chain variable region comprises SEQ ID NO:52, 53 or an amino acid sequence at least 70%, 80%, 90%, 95% or 99% identity to SEQ ID NO:52 or 53;
(i) the heavy chain variable region comprises SEQ ID NO:54 or an amino acid sequence at least 70%, 80%, 90%, 95% or 99% identity to SEQ ID NO:54; the light chain variable region comprises SEQ ID NO:57 or an amino acid sequence at least 70%, 80%, 90%, 95% or 99% identity to SEQ ID NO:57;
(j) the heavy chain variable region comprises SEQ ID NO:55 or an amino acid sequence at least 70%, 80%, 90%, 95% or 99% identity to SEQ ID NO:55; the light chain variable region comprises SEQ ID NO:58 or an amino acid sequence at least 70%, 80%, 90%, 95% or 99% identity to SEQ NO:58;
(k) the heavy chain variable region comprises SEQ ID NO:56 or an amino acid sequence at least 70%, 80%, 90%, 95% or 99% identity to SEQ ID NO:56; the light chain variable region comprises SEQ ID NO:59 or an amino acid sequence at least 70%, 80%, 90%, 95% or 99% identity to SEQ ID NO:59;
(l) the heavy chain variable region comprises SEQ ID NO:96 or an amino acid sequence at least 70%, 80%, 90%, 95% or 99% identity to SEQ ID NO:96; the light chain variable region comprises SEQ ID NO:59, 101, 102, 103 or an amino acid sequence at least 70%, 80%, 90%, 95% or 99% identity to SEQ ID NO:59, 101, 102 or 103;
(m) the heavy chain variable region comprises SEQ ID NO:97 or an amino acid sequence at least 70%, 80%, 90%, 95% or 99% identity to SEQ ID NO:97; the light chain variable region comprises SEQ ID NO:59 or an amino acid sequence at least 70%, 80%. 90%, 95% or 99% identity to SEQ ID NO:59;
(n) the heavy chain variable region comprises SEQ ID NO:98 or an amino acid sequence at least 70%, 80%, 90%, 95% or 99% identity to SEQ ID NO:98; the light chain variable region comprises SEQ ID NO:103 or an amino acid sequence at least 70%, 80%. 90%, 95% or 99% identity to SEQ ID NO:103;
(o) the heavy chain variable region comprises SEQ ID NO:99, 100 or an amino acid sequence at least 70%, 80%, 90%, 95% or 99% identity to SEQ ID NO:99 or 100; the light chain variable region comprises SEQ ID NO:57 or an amino acid sequence at least 70%, 80%, 90%, 95% or 99% identity to SEQ ID NO:57.

The antibody of the invention further comprises a coupling moiety linked to the polypeptide, the coupling moiety is selected from the group consisting one or more of radionuclides, drugs, toxins, cytokines, enzymes, fluorescein, carrier proteins, lipids, and biotin, wherein the polypeptide or antibody is selectively linked to the coupling moiety by a linker, preferably the linker is a peptide or polypeptide.

Wherein the antibody is selected from monoclonal antibodies, polyclonal antibodies, antisera, chimeric antibodies, humanized antibodies, and human antibodies.

Wherein the antibody is selected from multi-specific antibodies, single-chain variable fragments (says), single-chain antibodies, anti-idiotype (anti-Id) antibodies, diabodies, minibodies, nanobodies, single domain antibodies, Fab fragments, F(ab′) Fragments, disulfide-linked bispecific Fv (sdFv) and intracellular antibodies.

In another aspect, the invention discloses an antigenic epitope peptide, wherein the antigenic epitope peptide is derived from SEMG2 protein, and the amino acid of the antigenic epitope peptide comprises an amino acid sequence selected from the group consisting of SEQ ID NO:2 (QIEKLVEGKS), SEQ ID NO:3 (QIEKLVEGKSQIQ), SEQ ID NO:4 (QIEKLVEGKSQ), and SEQ ID NO:5 (QIEKLVEGKSQI).

The invention discloses a protein, wherein the protein comprises an amino acid sequence as the amino acid sequences shown in SEQ ID NO:2 (QIEKLVEGKS), SEQ ID NO:86 (QIEKLVEGKS(x)I(x)), SEQ ID NO:87 (QIEKLVEGKS(x))I), or SEQ ID NO:88 (QIEKLVEGKS(x)), preferably the polypeptide comprises an amino acid sequence of SEQ ID NO:3 (QIEKLVEGKSQIQ), SEQ ID NO:4 (QIEKLVEGKSQ), or SEQ ID NO:5 (QIEKLVEGKSQI) or an amino acid sequence at least 90% identity to any one of SEQ ID NOs:2-5, more preferably SEQ ID NOs:89-94 and SEQ ID NO:3 (corresponding to P1-P6, and P7 respectively); and a tag sequence which can selectively be linked at the N-terminus or C-terminus. Those skilled in the art should understand that the addition of a protein tag will not affect the prepared antibody's participation in binding between SEMG2 and CD27, the protein tag includes, but is not limited to, C-Myc, His, GST (glutathione S-transferase), HA, MBP (maltose-binding protein), Flag, SUMO, eGFP/eCFP/eYFP/mCherry, etc.

In one specific embodiment, the polypeptide has amino acid sequence of SEQ ID NO:3 (P7: QIEKLVEGKSQIQ) or SEQ ID NO:93 (P5).

The invention also discloses a method of preparing an antibody or an antigen-binding fragment thereof, wherein the protein is used as an immunogen to inject a subject such as a mouse or screening natural library to prepare the antibody, and the amino acid sequence of the antibody comprises an amino acid sequence of SEQ ID NO:2 (QIEKLVEGKS), SEQ ID NO: 86 (QIEKLVEGKS(x)I(x)), SEQ ID NO:87 (QIEKLVEGKS(x)I), or SEQ ID NO:88(QIEKLVEGKS(x)), preferably the polypeptide comprises an amino acid sequence of SEQ ID NO:3 (QIEKLVEGKSQIQ), SEQ ID NO:4 (QIEKLVEGKSQ), or SEQ ID NO:5 (QIEKLVEGKSQI) or an amino acid sequence at least 90% identity to any one of SEQ ID NOs:2-5, more preferably SEQ ID NO:93 (P5) or SEQ ID NO:3 (P7).

In a preferred embodiment, a method of obtaining isolated antibodies is disclosed, which uses a key epitope polypeptide of the binding between SEMG2 and CD27 as immunogen and screens with murine hybridoma and phage display in human and camel natural library.

In another aspect, the invention discloses an isolated polynucleotide encoding the compound, antigenic peptide, or protein.

The invention discloses a recombinant vector comprising the polynucleotide and optional regulatory sequences; preferably, the recombinant vector is a cloning vector or an expression vector.

Wherein, the regulatory sequence is selected from a leading sequence, a polyadenylation sequence, a polypeptide sequence, a promoter, a signal sequence, a transcription terminator, or any combination thereof.

The invention discloses a host cell comprising the recombinant vector.

Wherein, the host cell is a prokaryotic cell or a eukaryotic cell.

The invention discloses a pharmaceutical composition comprising the compound, the antigenic peptide, the protein, the polynucleotide, the recombinant vector, and one or more types of the host cells as previously described.

Wherein, the composition further comprises a pharmaceutically acceptable carrier or adjuvant.

The invention also discloses the use of the compounds, the antigenic peptide, the protein, the polynucleotide, the recombinant vector, or the host cell in preparation of products for agonizing or antagonizing the interaction between SEMG2 and CD27, preferably SEMG2 is expressed in tumor cells and CD27 is expressed in immune cells.

The invention also discloses the use of the compound, the antigenic peptide, the protein, the polynucleotide, the recombinant vector, or the host cell in preparation of a drug for preventing or treating tumors or a drug for modulating an immune response elicited against tumors.

In one specific embodiment, wherein the tumor is selected from one or more of colorectal cancer, lung cancer, melanoma, lymphoma, liver cancer, head and neck cancer, stomach cancer, kidney cancer, bladder cancer, prostate cancer, testicular cancer, endometrial cancer, breast cancer and ovarian cancer.

The invention also discloses a method of screening drugs or reagents preventing or treating tumors, comprising obtaining candidate drugs or reagents by screening inhibitors or antibodies which inhibit the interaction between SEMG2 and CD27.

The invention also discloses a method of preventing or treating tumors, comprising:

contacting immune cells such as lymphocytes (T lymphocytes) or tumor cells of the subject with an effective dose of any one of the compound; wherein the expression of SEMG2 in tumor cells can be selectively detected before contacting immune cells such as lymphocytes and/or tumor cells of the subject with an effective dose of the compound.

Wherein the subject has received or is receiving or will receive additional anti-cancer therapy.

Wherein the additional anti-cancer therapy comprises surgery, radiotherapy, chemotherapy, immunotherapy, or hormone therapy.

The invention also discloses a kit comprising one or more of the compounds, the antigenic peptides, the proteins, the polynucleotides, the recombinant vectors, and one or more types of the host cells, and the above components are accommodated in a suitable container.

The invention also discloses a method of detecting the presence or absence of SEMG2 in a biological sample in vitro, comprising: contacting the biological sample with the compound.

A method of inhibiting tumor cell growth of tumor cells, comprising the following steps: A) analyzing the expression of SEMG2 in tumor cells; B) contacting the tumor cells with an antibody recognizing SEMG2, the binding of the antibody to SEMG2 is KD<2×10⁻⁸; C) contacting T lymphocytes, with the antibody and tumor cells. Wherein the KD<2×10⁻⁸, <1×10⁻⁸, <9×10⁻⁹, <8×10⁻⁹, <7×10⁻⁹, <6×10⁻⁹, <5×10⁻⁹, <4×10⁻⁹, <3×10⁻⁹, <2×10⁻⁹, <1×10⁻⁹, <1×10⁻¹⁰.

### DESCRIPTION OF THE DRAWINGS

FIG. 1 depicts the result of co-immunoprecipitation assay, divided into upper panel and lower panel. The upper panel demonstrates the existence of physical interaction between human CD27 and SEMG2 (Flag). The lower panel demonstrates the existence of physical interaction between mouse CD27 and SEMG2 (Flag).

FIG. 2 depicts the results of immunofluorescent staining and ELISA. FIG. 2(A) demonstrates significant colocalization between CD27 and SEMG2 after overexpression in tumor cells. FIG. 2(B) shows the result of ELISA, demonstrating the effect of CD27 concentration-dependent on binding SEMG2 in microplate, while CD27 does not bind to the negative control protein and there is no concentration effect.

FIG. 3 depicts the result of co-immunoprecipitation assay for examining whether SEMG2 fragment (i.e., P1 to P6) binds to CD27. Wherein the P5 fragment has been detected with significant binding to CD27.

FIG. 4 depicts the result of co-immunoprecipitation assay for examining whether SEMG2 fragment (i.e., P4, P5, P6, P7) binds to CD27. Wherein the P7 sequence is derived from a part of P5, which is “QIEKLVEGKSQIQ”. The result includes left panel and right panel. The left panel shows the binding between the SEMG2 fragment and human CD27, and the right panel shows the binding between the SEMG2 fragment and mouse CD27. The results show that both human and mouse CD27 could bind to P5 and P7 fragments.

FIG. 5 depicts the contribution of each amino acid of P7 to binding CD27 protein, accurately demonstrated by Alanine Scanning method, including panel A and panel B. Panel A shows the sequence produced by substitution of each amino acid of P7 for glycine one by one, i.e., the mutated amino acid sequences numbered 1-13. Panel B is the result of co-immunoprecipitation experiment, indicating the extent to which the GFP fusion protein of mutant 1-1:3 polypeptide binds to CD27; wherein mutants 5 and 9 completely lost the binding to CD27; mutants 11 and 13 did not affect the binding between SEMG2 (497-509) and CD27; mutants at other sites (1, 2, 3, 4, 6, 7, 8, 10, 12) somewhat attenuated the binding between SEMG2 (497-509) and CD27. The amino acids located at positions 497, 498, 499, 500, 501, 502, 503, 504, 505, 506 and 508 of SEMG2 have obvious effects on the binding to CD27.

FIG. 6 depicts the binding between SEMG2 epitope polypeptides and CD27, and its competitive inhibition of full-length SEMG2 binding to CD27. (A) FIG. 6A shows BSA-conjugated human SEMG2 (497-509) polypeptide and monkey SEMG2 polypeptide can bind to CD27 protein on the microplate respectively, which are significantly higher than that of the negative BSA control, therefore indicating that CD27 can bind to both human and monkey SEMG2 (497-509) fragment. (B) FIG. 6B shows the inhibitory effect of SEMG2- derived polypeptides and derivatives QIEKLVEGKSQIQ, QIEKLVEGKSQI, QIEKLVEGKSQ and QIAKLVEGKSQ on the binding between full-length SEMG2 and CD27. As shown in the figure, different concentrations of peptides are firstly co-incubated with CD27-Fc for binding, and then added into the micro-reaction plate pre-coated with SEMG2 protein. After co-incubation, the unconjugated molecules are washed off, and anti-Fc secondary antibody-HRP are then used for detecting and developing. The results show that the polypeptide molecule can inhibit the binding between full-length SEMG2 and CD27.

FIG. 7 depicts apoptosis assay of HCT 116 cells stably transfected with SEMG2 or control empty vector and co-cultured with activated human peripheral blood mononuclear cells. (A) FIG. 7A is a representative image of apoptosis analysis, green field shows apoptotic cells, (B) FIG. 7B is based on the statistics of three independent biological experiments (error bars represent standard deviation).

FIG. 8 depicts immunoblotting assay to show the expression of SEMG2 protein in different tumor cells. The names of the tumor cells are indicated above (the font is tilted 45 degrees). About half of the tested cell lines have detectable SEMG2 protein expression.

FIG. 9 depicts the result of immunohistochemistry (IHC) assay revealing the expression of SEMG2 protein in different tumor tissues. (A) FIG. 9A shows the expression of SEMG2 in different colorectal cancer tumor tissues, with normal colorectal tissue as control; (B) FIG. 9B shows the expression of SEMG2 in different lung cancer tissues, with normal lung tissue as control; (C) FIG. 9C shows representative images of SEMG2 positive expression in prostate cancer, melanoma, and gastric cancer. Due to space limitation, the detection results of all tumor types are not listed here in detail; (D) FIG. 9D is the rate of SEMG2 positive expression among different types of tumors. Positive expression is defined as moderate or strong positive expression by immunohistochemical staining. Statistical results based on tissue chips (each chip included more than 50 tissue samples) are plotted as percentages to show the positive expression ratio of SEMG2.

FIG. 10 depicts the statistical result of the Kaplan-Meier factor survival analysis, which suggests that high expression of SEMG2 (defined as moderate, strong positive staining by SEMG2 immunohistochemical staining) is significantly associated with shortened overall survival in colorectal cancer patients. P value below 0.001 indicates a highly significant association.

FIG. 11 depicts the result of the immunohistochemical assay. The upper panel shows the statistical result of the correlation between the staining of regulatory T lymphocytes, namely Treg and SEMG2 in lung cancer. The intensity of SEMG2 immunohistochemical staining is divided into different levels, and the number of Treg (labeled with Foxp3 antibody) in each field of view is counted separately and compared. The bottom panels are Treg marked representative images of SEMG2 positive and negative expression, respectively.

FIG. 12 depicts the result of ELISA. The ordinate shows the normalized A405 absorbance value as the reading of ELISA, showing the degree of binding between SEMG2 and CD27; the abscissa shows the concentration of antibody added. The solid line represents the blocking effect of the polyclonal antibody generated by SEMG2(497-509) as an antigen; the dotted line represents the blocking effect of the polyclonal antibody generated by the full-length protein of SEMG2 as an immunogen. The polyclonal antibody generated by SEMG2(497-509) requires a lower concentration to exert the blocking effect, that is, the blocking titer of the antibody generated by SEMG2(497-509) is higher than that of the SEMG2 full-length protein. It suggests that the recognition of the key role epitope, SEMG2(497-509), makes the development of blocking antibodies much easier.

FIG. 13 depicts the number of blocking monoclonal antibodies and the total number of antibodies obtained after injecting mice with SEMG2 (497-509) epitope peptide and full-length SEMG2 protein as immunogens. Among the mouse monoclonal antibodies obtained by hybridoma fusion, the antibodies confirmed by ELISA that can inhibit the binding between SEMG2 and CD27 are counted and displayed as black bars. Most of the antibodies prepared from SEMG2 (497-509) epitope fragment as immunogen can block the binding between SEMG2 and CD27, and the positive rate is significantly higher than that of antibodies prepared using full-length SEMG2 as immunogen.

FIG. 14 depicts the binding ability of the mouse monoclonal antibody and the humanized mouse monoclonal antibody to SEMG2 protein. The reading of the ELISA, the OD₄₅₀ absorbance, is used as the ordinate, and the abscissa shows the different concentrations of antibody added. As the concentration of the antibody in the ELBA system increases, the OD₄₅₀ value gradually increases, indicating that the binding of SEMG2 to the murine monoclonal antibody (FIG. 14A) or the humanized monoclonal antibody (FIG. 14B) increases gradually. The fitting curve is a representative result based on statistics from three independent biological experiments.

FIG. 15 depicts the ability of mouse monoclonal antibody in binding to BSA-SEMG2 (497-509) and blocking the binding between SEMG2 and receptor protein. (A) Panel A shows the reading of the ELISA, the OD₄₅₀ absorbance, is used as the ordinate, and the abscissa shows the added antibody with different concentrations, which indicates a gradually increased binding between SEMG2 (497-509) and the mouse monoclonal antibody. (B) Panel B shows that mouse monoclonal antibody blocks the binding between SEMG2 and CD27, and the blocking effect increases with the increasing concentration. The control mouse IgG antibody does not show blocking function. The ordinate is the blocking ratio which is the normalized blocking ratio; the abscissa shows the different concentrations of antibody added. The binding between SEMG2 and CD27 gradually decreased as the antibody concentration increased in the ELISA system. The fitting curve is based on statistics of three independent biological experiments (error bars represent standard deviation).

FIG. 16 depicts the result of ELISA. The ordinate shows the normalized OD₄₅₀ absorbance as reading of the ELISA, which indicates the extent of binding between SEMG2 (fixed on the surface of ELBA plate) and CD27-Fc added; the abscissa shows different experimental conditions, i.e., different antibodies co-incubated (the concentration is 10 μg/mL): HPA042767 and HPA042835 are rabbit polyclonal antibodies against SEMG2(354-403) and SEMG2(563-574) respectively; MM02, MM05, MM07, MM08, MM13, MM14 are mouse monoclonal antibodies against SEMG2(497-509) epitope. The results show that mouse monoclonal antibodies against SEMG2(497-509) epitope, but not antibodies against other epitopes, block the binding between SEMG2 and CD27. This experiment demonstrates that the mouse monoclonal antibodies against SEMG2(497-509) epitope functionally belong to the same type of antibodies.

FIG. 17 depicts the effect of different types of antibodies on tumor cell killing effect by T cells. Activated human peripheral blood monocytes (PBMC) are co-cultured with human melanoma cell A375 highly expressing SEMG2 or colorectal cancer cell LOVO respectively, and meanwhile different antibodies are added: irrelevant mouse IgG, HPA042767, HPA042835, MM02, MM05, MM07, MM08, MM13, or MM14. The ordinate shows the percentage of apoptotic tumor cells; the abscissa shows different treatment conditions in experiment, i.e., the different antibodies added. Mouse monoclonal antibodies (MM02, MM05, MM07, MM08, MM13, or MM14) against SEMG2(497-509) epitope significantly promote the tumor killing effect by T cells, while the control irrelevant IgG or HPA042767 and HPA042835 antibodies against SEMG2(354-403) and SEMG2(563-574) antigenic epitopes do not show such a function. It demonstrates that SEMG2(497-509) epitope, are key sites of SEMG2 expressed by tumor cells in immune escape function, and the antibodies directed against this epitope belong to the same type in terms of anti-tumor immunomodulatory function.

FIG. 18 depicts the result of T cell killing experiment to different tumor cells in presence of SEMG2 blocking antibodies. Wherein A375 and LOVO are tumor cells highly expressing SEMG2 protein, while DLD1, NCM460 and NCI-H1975 are SEMG2-negative cells. During the T cell killing experiment on the tumor cells, different antibodies are added, i.e.: irrelevant murine IgG antibody, MM02 or MM05 mouse monoclonal antibody. The abscissa represents the different tumor cell lines, while the ordinate represents the percentage of apoptotic tumor cells. Tumor cells with higher expression of SEMG2 (A375 and LOVO) can be more effectively killed by T cells after antibody treatment, while there is no obvious increase in apoptosis level of tumor cells without SEMG2 expression (DLD1, NCM460 and NCI-H1975) after administration of SEMG2 blocking antibodies MM02 and MM05. This demonstrates that positive expression of SEMG2 can be used as a selective marker for administration of SEMG2-blocking antibodies. When SEMG2 blocking antibody is used as an anti-tumor immune drug, the expression of SEMG2 has guiding significance for the selection of suitable patients.

FIG. 19 depicts the A450 absorbance as a reading in ELBA to detect the extent of SEMG2 binding to different antibodies. Different antigens from SEMG2 (shown on the left) were coated on the ELISA plates and conjugated with HPA04276, HPA042835, MM02, MM05, MM07, MM08, MM13, MM14, followed by bound antibody detection using anti-mouse secondary antibody (against HPA04276, HPA042835, MM02, MM05, MM07 and MM08) or anti-rabbit secondary antibody (against HPA04276, HPA042835). MM02, MM05, MM07, MM08, MM13, and MM14 all bind to the SEMG2 (497-509) epitope and belong to the same type; HPA04276 binds to the SEMG2 (354-403) epitope, and HPA042835 binds to the SEMG2 (563-574) epitope.

FIG. 20 depicts the value detected by ELISA, i.e., OD₄₅₀ absorbance. In this experiment, the SEMG2 (497-509) epitope peptide and its glycine scan mutant (i.e., amino acid substitution to glycine one by one) polypeptides are immobilized on an ELISA plate, and further bind to different antibodies as shown in figure. This experiment is used to determine the precise amino acid epitopes that different monoclonal antibodies bind to, and the relative importance of each amino acid to the binding antibody. The control antibodies HOA04276 and HPA042835 do not bind to the epitopes and mutants; the amino acids at different sites contribute differently to the binding of the antibodies; and important amino acids bound by each antibody (MM02, MM05, MM07, MM08, MM13, MM14) which blocks the binding between SEMG2 and CD27 are similar. This demonstrates that antibodies with blocking function belong to the same type in terms of binding epitopes.

FIG. 21 depicts the result of ELISA, which shows effects of fully human antibodies H88-67, H88-93, H88-96 and affinity mature fully human antibodies concentration-dependent on binding to SEMG2 and BSA-SEMG2(497-509) polypeptide. The reading of the ELISA, the OD₄₅₀ absorbance, is used as the ordinate, and the abscissa shows the different concentrations of antibody added.

FIG. 22 depicts the result of ELISA shows effects of the fully human antibody and mouse monoclonal antibodies concentration-dependent on competitively binding to SEMG2. The ordinate shows the ratio of fully human antibody blocking the binding between SEMG2's and mouse antibodies. As the concentration of fully human antibody increases, the detected signal of mouse antibodies binding to SEMG2 gradually decreases.

FIG. 23 depicts the result of ELISA, which shows the effect of different human antibodies H88-93, H88-96 and H88-67 blocking the binding between SEMG2 and CD27. All antibody concentrations are 10 μg/mL. Antibody clones H88-93, H88-96 and H88-67 are all fully human antibodies screened in the natural phage library using the SEMG2 (497-509) epitope.

FIG. 24 depicts the degree of killing of co-cultured A375 and LOVO tumor cells by T cells, and the influence of human antibodies H88-93, H88-96, and H88-67 on the killing effect. The result demonstrates that the three antibodies against SEMG2 (497-509) epitope significantly promote killing SEMG2-expressing tumor cells by T cells.

FIG. 25 depicts the binding between SEMG2 and fully human antibody molecules determined by Bio-Layer Interferometry shows the changes in the binding and dissociation of fully human antibodies in solution to the SEMG2 protein molecules immobilized on the biosensor, based on which the affinity constant between the fully human antibody and SEMG2 is calculated.

FIG. 26 depicts the SEMG2 antibody significantly inhibits tumor growth in the A375 melanoma mouse in vivo model.

FIG. 27 depicts the phenotypic analysis results of the homozygous knockout of the mouse gene Svs3a corresponding to human SEMG2 compared to wild-type mice, including specific results of gross morphology, biopsy examination of various tissues and organs, ratio analysis of different subtypes of T lymphocytes, blood biochemistry, liver function and routine blood tests.

### DETAILED DESCRIPTION

The following makes further explanations to the invention by detailed description.

Unless defined otherwise, all technical and scientific terms used herein have the same meaning as commonly understood by one of ordinary skill in the art to which the invention pertains.

In this application, the singular forms “a”, “an” and “the” include plural reference, unless the context clearly dictates otherwise.

As used herein, the term “subject” includes any human or nonhuman animals. The term “nonhuman primate” includes all vertebrates, such as mammals or nonmammals, for example nonhuman primates, sheep, canines, felines, equines, bovines, chickens, rats, mice, amphibians, reptiles, and the like. Unless otherwise specified, the terms “patient” and “subject” can be used interchangeably. In the present invention, a subject is preferably human.

As used herein, the term “SEMG2” is human semenogelin 2, one of the major components in human semen, secreted by seminal gland, and forms colloidal material to coat sperm cells and restrict their movement. The proteolytic enzymes and fibrinolytic enzymes secreted by the prostate gland in semen can break down the semenogelin and promote semen liquefaction, allowing sperm to move more freely. See Yoshida K, Karzai Z T, Krishna Z, Yoshida M, Kawano N, Yoshida M, et al., Cell Motil Cytoskeleton. 2009; 66(2):99-108. The “SgII A” polypeptide isolated from SEMG2 protein has antibacterial activity, and the sequence is H-KQEGRDHDKSKGHFHMIVIHHKGGQAHHG-OH. It should be noted that different from the key amino acid sequence for binding between SEMG2 and CD27 described in the present invention, the antimicrobial peptide sequence is located in a completely different region of SEMG2. See Edström A M, Maim J, Frohm B, Martellini J A, Giwercman A, Mörgelin M, et al., J Immunol. 2008; 181(5):3413-21. In addition, SEMG2 has also been reported to bind to zinc ions and affect the activity of prostatic proteolytic enzyme PSA. See Jonsson M, Linse S, Frohm B, Lundwall A, Malm J. Biochem J. 2005; 387(Pt 2):447-53.

As used herein, the term “antibody” includes intact antibody and any antigen-binding fragment (i.e., “antigen-binding part”) or the single chain thereof. “Antibody” refers to a protein containing at least two heavy (H) chains and two light (L) chains connected by disulfide bond, or its antigen-binding part. Each heavy chain consists of a heavy chain variable region (short for VH herein) and a heavy chain constant region. The heavy chain constant region consists of three domains, CH1, CH2 and CH3. Each light chain consists of a light chain variable region (short for VL herein) and a light chain constant region. The light chain constant region consists of a CL domain. The VH and VL regions can be further subdivided into high-variable regions, known as complementary decision area (CDR), scattered over more conservative regions known as framework region (FR). Each VH and VL consists of three CDRs and four FRs arranged in the following order from the amino terminus to the carboxy terminus: FR1, CDR1, FR2, CDR2, FR3, CDR3, FR4. The variable regions of heavy and light chains contain binding domains for antigen interaction.

The term “antibody” refers to immunoglobulins or their fragments or derivatives thereof, and includes any polypeptides that contain antigen binding sites, whether they are produced in vitro or in vivo. The term includes, but is not limited to, multi-clone, monoclonal, monospecific, multispecific, nonspecific, humanized, single-chain, chimeric, synthetic, recombinant, hybridized, mutation, and graft antibodies. The term “antibody” also includes antibody fragments such as Fab, F(ab′) 2, Fv, scFv, Fd, dAb, and other antibody fragments that retain antigen binding function, i.e., can specifically bind to PD-1. Generally, such fragments will contain antigen binding fragments.

The terms “antigen-binding fragment”, “antigen-binding domain” and “binding fragment” refer to an antibody molecule, which contains amino acids responsible for the binding between specific antibodies and antigens. For example, where the antigen is large and the antigen-binding fragment binds only a portion of the antigen. That is, the part of the antigen molecule responsible for the specific interaction with the antigen binding fragment is called “epitope” or “antigenic determinant”.

An antigen-binding fragment typically comprises an antibody light chain variable region (VL) and an antibody heavy chain variable region (VH), however, it does not necessarily have to comprise both. For example, a so-called Fd antibody fragment consists only of a VH domain, but still retains some of the antigen binding functions of the intact antibody.

The term “epitope” is defined as an antigenic determinant which specifically binds/recognizes a binding fragment. Binding fragments can specifically bind/react with a conformation that is unique to the target structure or a contiguous epitope, the conformation or discontinuous epitope is characterized by that the polypeptide antigen being two or more separated discrete amino acid residues in the primary sequence, but the polypeptides are aggregated together on the surface of the molecule when they are folded into native proteins/antigens. Two or more discrete amino acid residues of an epitope exist in separate parts of one or more polypeptide chains. When the polypeptide chain folds into a three-dimensional structure, these residues gather on the surface of the molecule to form an epitope. In contrast, contiguous or linear epitopes, consisting of two or more discrete amino acid residues, are present in a single linear segment of a polypeptide chain.

The terms “treating” or “treatment” refer to both therapeutic treatment and prophylactic/preventing measures. Those in need of treatment include individuals who already have a particular medical condition, as well as those who may eventually acquire the condition.

The term “vector” as used herein refers to a molecular tool for the transport, transduction, and expression in a target cell of a contained exogenous gene of interest (for example, a polynucleotide according to the present invention). The tool provides a suitable nucleotide sequence that initiates transcription, i.e., the promoter.

The terms “tag protein” and “protein tag” in the present invention are interchangeable, and refer to a polypeptide or protein fused and expressed with the target protein by using DNA in vitro recombination technology, to facilitate protein expression, detection, tracking and purification. Tag proteins include, but not limited to, His6, Flag, GST, MBP, HA, GFP and Myc.

### EXAMPLES

Unless otherwise specifically explained, the implementation methods in the following examples are all conventional method. The invention will be further understood with reference to the following non-limiting experimental examples.

### Example 1: Detection of Binding Between SEMG2 and CD27

Human HEK293 cells were co-transfected in a 10 cm diameter culture dish. 48 hours after co-transfection with complex including pcDNA3-Flag-SEMG2 plasmid and pcDNA3-HA-CD2 plasmid, cells were collected and lysed, CD27 in lysate was enriched by standard immunoprecipitation procedure. The antibody used for immunoprecipitation was Flag antibody, and IgG nonspecific antibody was used for the control group. Immunoblotting (western blot) experiment was carried out later using HA antibody to detect the amount of co-immunoprecipitated CD27 and using Flag antibody to detect the amount of immunoprecipitated SEMG2. In immunoblotting assay, cells were lysed by Roche Complete protease inhibitor in 1% Triton X-100 (TBS pH7.6) for 30 minutes on ice, and insoluble material was pelleted by centrifugation. Lysate in SDS sample buffer with 50 mM DTT was heated to 100° C. for 10 minutes, separated by SDS-PAGE and transferred to PVDF membrane (Millipore). The cell membrane was blocked in TBS with 5% bovine serum albumin (BSA) and probed with the indicated antibodies. The bands were visualized with West Pico (Thermo Fisher Scientific).

In co-immunoprecipitation experiment, cells were lysed in IP buffer (Thermo Scientific) and Roche complete protease inhibitor for 10 minutes, followed by the addition of benzonase (sigma) for 25 minutes at room temperature. The lysate was then centrifuged at 15,000 rpm at 4° C. to remove the precipitate. The supernatant was then incubated with primary antibody slowly rotated overnight at 4° C., followed by the addition of protein A or protein G dynabeads and incubated at 4° C. for 2 hours, washed for 4 times in PBST (PBS with 0.01% Tween 20), eluted with 50 mM DTT in SDS sample buffer for 10 minutes at 100° C., separated with SDS, and immunoblotted as previously described.

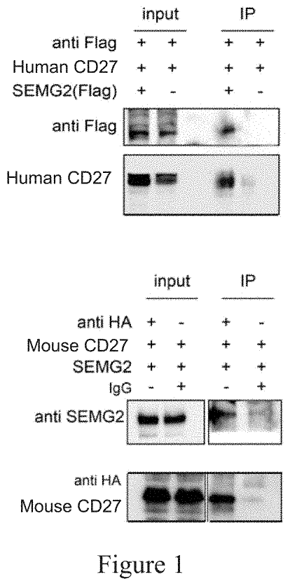

The result showed that precipitation with Flag antibody made the precipitated complex contain both SEMG2 and CD27, while the control group did not contain SEMG2 or CD27. See FIG. 1 for the experimental result. This experiment indicates a physical interaction between SEMG2 and human CD27. In addition, the interaction between SEMG2 and mouse CD27 was detected using the same method described above. In this experiment, mouse CD27 was used instead of human CD27 for HEK293 cell transfection, and the other experimental conditions remained unchanged. The experimental result is shown in FIG. 1. This experiment indicates a physical interaction between SEMG2 and mouse CD27.

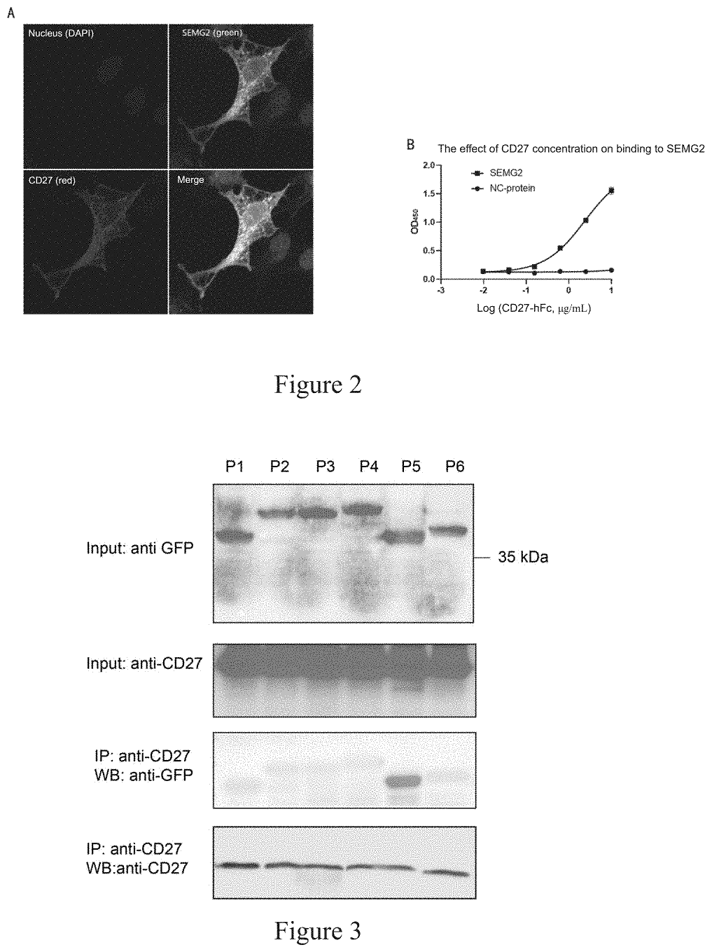

Under the above co-transfection experimental conditions, the pre-placed cell slides in a 10 cm dish were fixed, permeabilized, and blocked, and further immunolabeled with antibodies containing HA tag (mouse anti) and Flag tag (rabbit anti) simultaneously for CD27 and SEMG2, and then labeled with secondary antibodies to show red and green colors, respectively. The co-localization of SEMG2 and CD27 in cells was observed under fluorescence confocal microscopy. The results are shown in FIG. 2. The co-expressed SEMG2 and CD27 proteins showed obvious co-localization in cells, and the localization patterns were even nearly identical. This is consistent with the finding that the binding between SEMG2 and CD27 proteins.

### Example 2: Binding Between SEMG2(497-509) Fragment and CD27 Protein

To further confirm which part of SEMG-2 binds to CD27, fragments of SEMG2 protein were designed. The amino acid sequence of full-length SEMG2 protein (SEQ ID NO:1) was divided into 6 segments of sequences, fused with GFP and named as SEMG2-P1, SEMG2-P2, SEMG2-P3, SEMG2-P4, SEMG2-P5 and SEMG2-P6 (See Table 1 for specific sequences, with corresponding abbreviation as P1-P6 respectively). Plasmids expressing these amino acid sequences were co-transfected with CD27 into HEK293 cells, and co-immunoprecipitation experiments were performed to identify the main fragment of SEMG2 that binds to CD27. The co-immunoprecipitation results are shown in FIG. 3. Only SEMG2-P5 had significant binding to CD27, while SEMG2-P1 SEMG2-P2, SEMG2-P3, SEMG2-P4 and SEMG2-P6 did not bind to CD27. The above results indicated that the SEMG2-P5 fragment is the major part that binds to CD27.

(human SEMG2):
SEQ ID NO: 1
MKSIILFVLSLLLILEKQAAVMGQKGGSKGQLPSGSSQFPHGQKGQHYFG
QKDQQHTKSKGSFSIQHTYHVDINDHDWTRKSQQYDLNALHKATKSKQHL
GGSQQLLNYKQEGRDHDKSKGHFHMIVIHHKGGQAHHGTQNPSQDQGNSP
SGKGLSSQCSNTEKRLWVHGLSKEQASASGAQKGRTQGGSQSSYVLQTEE
LVVNKQQRETKNSHQNKGHYQNVVDVREEHSSKLQTSLHPAHQDRLQHGP
KDIFTTQDELLVYNKNQHQTKNLSQDQEHGRKAHKISYPSSRTEERQLHH
GEKSVQKDVSKGSISIQTEEKIHGKSQNQVTIHSQDQEHGHKENKISYQS
SSTEERHLNCGEKGIQKGVSKGSISIQTEEQIHGKSQNQVRIPSQAQEYG
HKENKISYQSSSTEERRLNSGEKDVQKGVSKGSISIQTEEKIHGKSQNQV
TIPSQDQEHGHKENKMSYQSSSTEERRLNYGGKSTQKDVSQSSISFQIEK
LVEGKSQIQTPNPNQDQWSGQNAKGKSGQSADSKQDLLSHEQKGRYKQES
SESHNIVITEHEVAQDDHLTQQYNEDRNPIST

TABLE 1
Corresponding amino acid sequences for construction of SEMG2 expression
fragments

| | | |
|---|---|---|
| SEMG2-P1 | GSFSIQHTYHVDINDHDWTRKSQQYDLNALHKATKSKQHLGGSQQLLNY | SEQ ID NO: 89 |
| | KQEGRDHDKSKGHFHMIVIHHKGGQAHHGT | |
| SEMG2-P2 | QNPSQDQGNSPSGKGLSSQCSNTEKRLWVHGLSKEQASASGAQKGRTQ | SEQ ID NO: 90 |
| | GGSQSSYVLQTEELVVNKQQRETKNSHQNKGHYQNVVDVREEHSSKLQT | |
| | SLHPAHQDRLQHGPKDIFTTQDELLVYNKNQHQTKNLSQDQEHGR | |
| SEMG2-P3 | KAHKISYPSSRTEERQLHHGEKSVQKDVSKGSISIQTEEKIHGKSQNQVTIHS | SEQ ID NO: 91 |
| | QDQEHGHKENKISYQSSSTEERHLNCGEKGIQKGVSKGSISIQTEEQIHGKS | |
| | QNQVRIPSQAQ | |
| SEMG2-P4 | EYGHKENKISYQSSSTEERRLNSGEKDVQKGVSKGSISIQTEEKIHGKSQNQ | SEQ ID NO: 92 |
| | VTIPSQDQEHGHKENKMSYQSSSTEERRLNY GGKSTQKDVSQSSIS | |
| SEMG2-P5 | FQIEKLVEGKSQIQTPNPNQDQWSGQNAKGKSGQSADSKQDLLSH | SEQ ID NO: 93 |
| SEMG2-P6 | EQKGRYKQESSESHNIVITEHEVAQDDHLTQQYNEDRNPIST | SEQ ID NO: 94 |
| SEMG2-P7 | QIEKLVEGKSQIQ | SEQ ID NO: 3 |

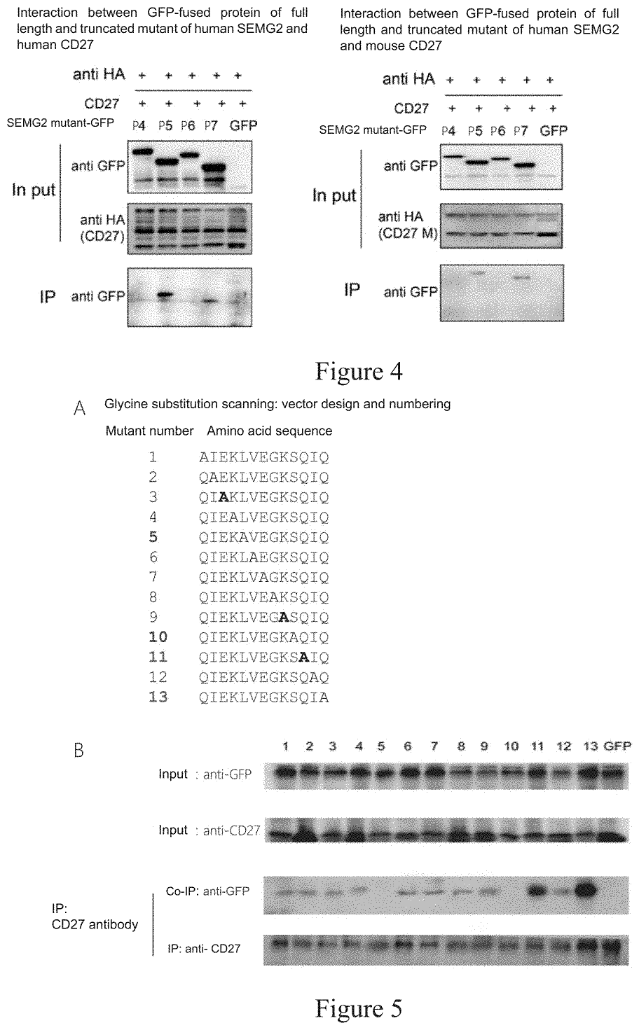

To further confirm the key amino acids in the binding between SEMG2-P5 sequence and CD27, SEMG2(497-509) fragment was selected and named as SEMG2-P7 (the specific sequence is QIEKLVEGKSQIQ, abbreviated as P7 or SP7). SEMG2-P7(497-509), SEMG2-P5(positive control), SEMG2-P4 (negative control) or SEMG2-P6 (negative control) was co-transfected with CD27 into HEK293 cells respectively, including human CD27 and mouse CD27. The result of co-immunoprecipitation experiment performed later showed that both SEMG2-P7 and SEMG2-P5 bind to CD27, and the results of human CD27 and mouse CD27 were the same. The experimental results are shown in FIG. 4. This co-immunoprecipitation experiment confirmed that SEMG2(497-509) is the main structure that binds to human and mouse CD27.

### Example 3: Precise Characterization of the Key Amino Acids of SEMG2 Binding to CD27 Using Glycine Scanning Method

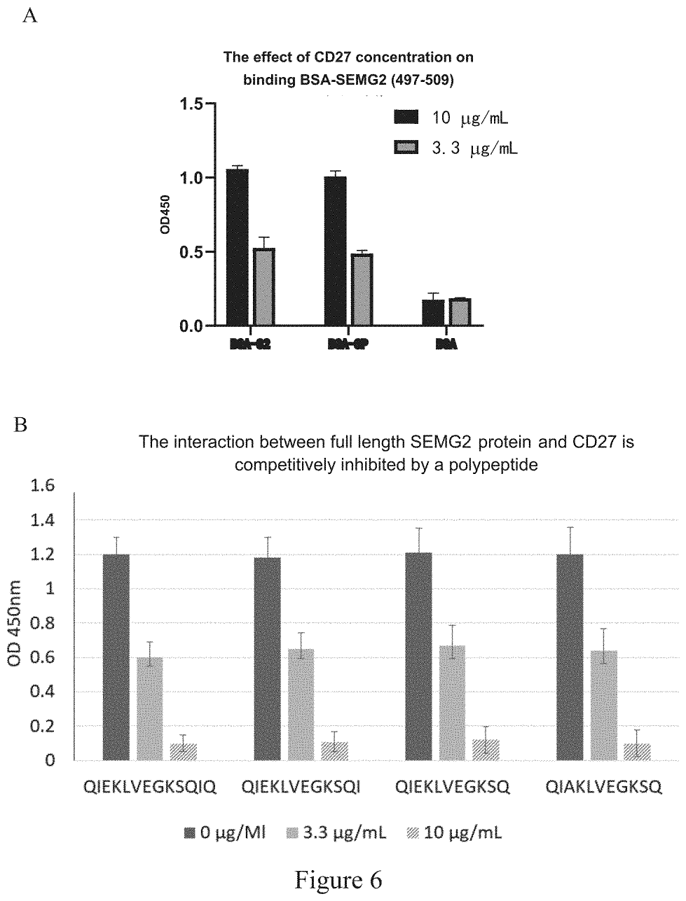

To characterize the epitope of SEMG2 binding to CD27 with higher resolution, and to demonstrate the contribution of each amino acid of SEMG2(497-509) to binding CD27 protein more accurately, each amino acid of SEMG2(497-509) was replaced one by one with glycine, and the resulting sequences are mutant amino acid sequences numbered 1-13 (see FIG. 5). These mutant plasmids and CD27 expression vector were co-transfected into HEK293 cells, and the degree of GFP-fused 1-13 polypeptide variants binding to CD27 was detected by co-immunoprecipitation assay. The experimental results are shown in FIG. 5, wherein mutants 5 and 9 completely lost the binding to CD27; mutants 11 and 13 did not affect the binding of SEM-2 (497-509) to CD27; mutants at other sites (1, 2, 3, 4, 10, 12) weakened the binding between SEMG2(497-509) and CD27 to some extent. Therefore, it can be seen that the amino acids at positions 497, 498, 499, 500, 501, 502, 503, 504, 505, 506 and 508 of SEMG2 have obvious effects on the binding to CD27. The peptide sequence 497-509 was coupled to BSA, and coated onto a 96-well microplate. CD27-hFc at different concentrations was used as the primary antibody to detect the ability of CD27 to bind to the peptide sequence. The experimental result is shown in FIG. 6. The result demonstrates that the effect of CD27 concentration on binding to SEMG2 does exist.

### Example 4: SEMG2 Expressed by Tumor Cells Inhibits Effect of Killing Tumors by Immune Cells

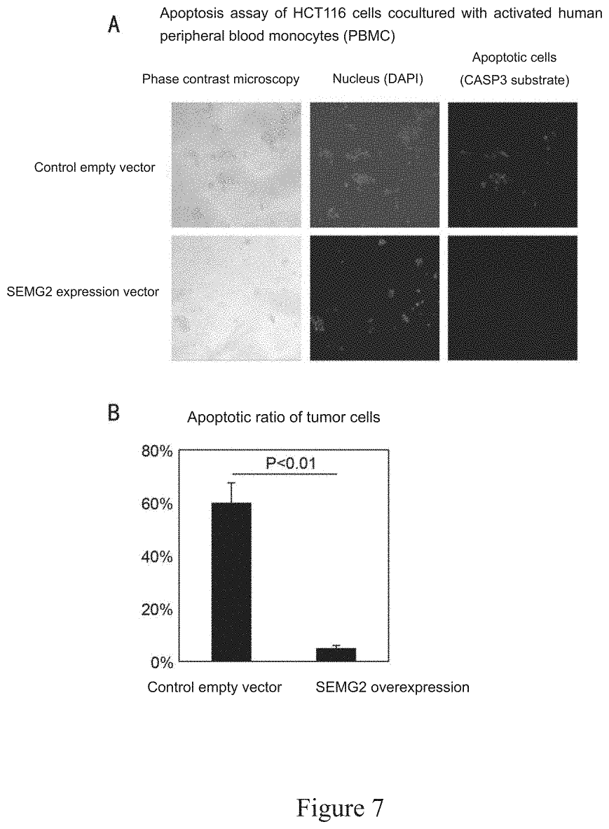

T cell-mediated killing assay of tumor cells. HCT116 human colorectal cancer cells were stably transfected with SEMG2 expression vector or control empty vector, and the proportion of apoptotic cells after co-culturing with activated PBMCs was determined by caspase3/7 lysis assay (green fluorescence assay). Specifically; HCT116 cells stably expressing SEMG2 were seeded in 96-well plate. Human peripheral blood mononuclear cells (PBMC; 470025, Stem Cell) were activated with 100 ng/mL of CD3 antibody, 100 ng/mL of CD28 antibody, and 10 ng/mL of IL2 (#317303; #302913; #589102, BioLegend) respectively, and co-cultured with the colorectal cancer cells (#4440, Essen Bioscience) at a ratio of 10:1 in presence of fluorescent caspase-3/7 substrate. After 10 hours, cells were observed under a fluorescence microscope. The result is shown in FIG. 7. Compared to control cells, tumor cells overexpressing SEMG2 exhibited significantly reduced apoptosis after co-culture with activated PBMCs. The result of this experiment supports that SEMG2 has a role in suppressing immune cell function.

### Example 5: Detection of SEMG2 Expression in Different Tumor Cells

Different types of human tumor cells, including LOVO colorectal cancer, RKO colorectal cancer, PC3 prostate cancer, A375 malignant melanoma, SW1116 colorectal cancer, DLD1 colorectal cancer, HEK293 human renal epithelial cell line, HepG2 hepatocellular carcinoma, NCM460 human normal colonic epithelial cells, NCI-H1975 human non-small cell lung adenocarcinoma, CaCo2 colonic adenocarcinoma, HT29 colorectal adenocarcinoma, SW1990 human pancreatic adenocarcinoma, AGS human gastric adenocarcinoma, SW480 colorectal cancer, SaOS2 osteosarcoma, GES-1 human gastric mucosal cells, and so like, were incubated with DMEM medium containing 10% calf serum in cell incubator with 5% carbon dioxide at 37° C.

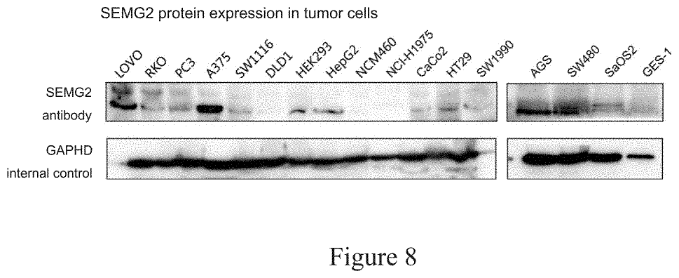

In immunoblotting assay, cells were lysed by Roche complete protease inhibitor in 1% Triton X-100 (TBS pH7.6) for 30 minutes on ice, and insoluble material was pelleted by centrifugation. Lysate in SDS sample buffer with 50 mM DTT was heated to 100° C. for 10 minutes, separated by SDS-PAGE and transferred to PVDF membrane (Millipore). The cell membrane was blocked in TBS with 5% bovine serum albumin (BSA) and probed with the primary antibodies specifically against SEMG2 and internal control GAPDH, respectively. The primary antibodies were labeled with HRP-conjugated secondary antibodies. The bands were visualized with West Pico (Thermo Fisher Scientific). The results are shown in FIG. 8, indicating that SEMG2 was not expressed in GES-1 human gastric mucosal cells and NCM460 human normal colonic epithelial cells, but observably expressed in multiple types of malignant tumor cells, including LOVO colorectal cancer, RKO colorectal cancer, PC3 prostate cancer, A375 malignant melanoma, SW1116 colorectal cancer, HEK293 human renal epithelial cell line, HepG2 hepatocellular carcinoma, CaCo2 colonic adenocarcinoma, HT29 colorectal adenocarcinoma, AGS human gastric adenocarcinoma, SW480 colorectal cancer and SaOS2 osteosarcoma. This result demonstrates that SEMG2 is a protein ubiquitously expressed in tumors.

### Example 6: Detection of SEMG2 Expression in Different Tumor Cells Using Immunohistochemistry (IHC)

For immunohistochemical staining, we obtained tissue chips of various tumors from Shanghai Xinchao Biotechnology Company. Briefly, tissue specimens were incubated with anti-SEMG2 antibody (HPA042767, purchased from Sigma Aldrich, 1:100 dilution) and a biotin-conjugated secondary antibody, followed by incubation with an anti-biotin-biotin-peroxidase complex, and observed with chromophoric reagent aminoethylcarbazole. As the histological score, staining intensities were divided into four groups: high (3), moderate (2), low (1), and negative (0).

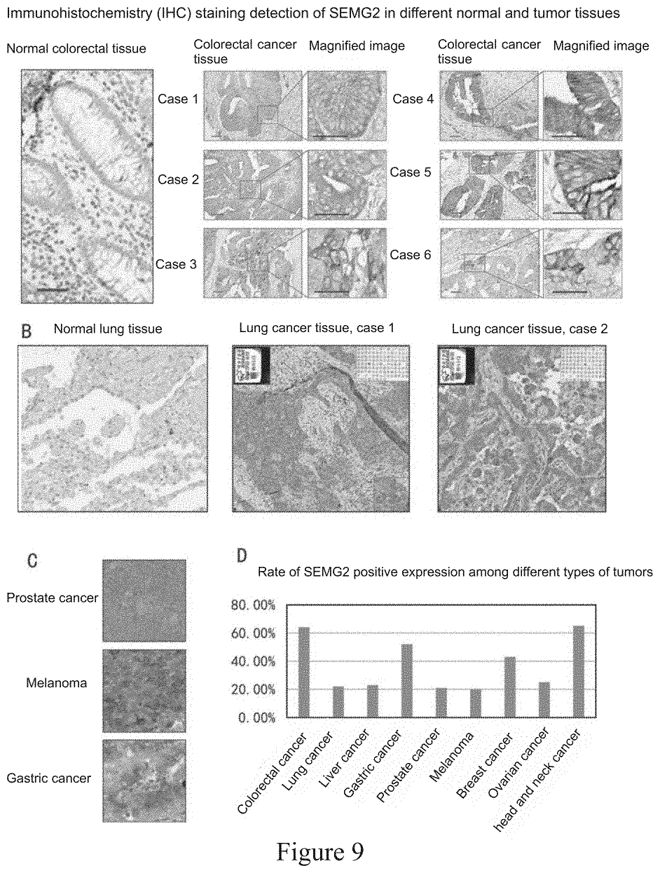

First, the expression of SEMG2 in tissue chips of colorectal cancer tumor was stained, and normal colorectal tissue was used as control; it was found that there was extensively high expression of SEMG2 in colorectal cancer tissue. The result is shown in FIG. 9.

Next, the expression of SEMG2 in different lung cancer tissues was stained, and normal lung tissue was used as control; it was found that there was extensively high expression of SEMG2 in lung cancer tissues. The results are shown in FIG. 9.

Again, the positive expression of SEMG2 in prostate cancer, melanoma, and gastric cancer was stained, and the results are shown in FIG. 9.

Finally, based on the tissue chip staining, the positive rate of SEMG2 expression in the different tumor types indicated was calculated. Positive expression was defined as moderate or strong positive expression in immunohistochemical staining. Statistical results based on tissue chips (each chip comprises more than 50 tissue samples) are shown as a percentage in FIG. 9.

### Example 7: Demonstration of the Association Between High SEMG2 Expression and Poor Tumor Prognosis

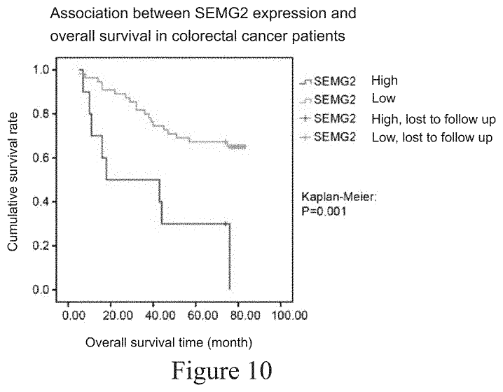

In immunohistochemical staining, we obtained tissue chips of various tumors from Shanghai Xinchao Biotechnology Co., Ltd., all with follow-up data of survival time information. Immunohistochemical detection was performed by the method described in Example 6. Taking colorectal cancer as an example, the patients were classified according to the expression of SEMG2, and divided into two groups: high SEMG2 (immunohistochemical score of 2, 3) and low SEMG2 (immunohistochemical score of 0, 1). The Kaplan-Meier method was used to compare the overall survival of the two patient groups. The results are shown in FIG. 10. It was found that the survival of patients with high SEMG2 expression was significantly shorter than that of tumor patients with low SEMG2 expression. There was also such significant correlation for other tumors such as lung cancer (P<0.05) and gastric cancer (P<0.05). The above results suggest that SEMG2 is a key molecule for tumor immune evasion and may serve as a new anti-tumor target.

### Example 8: Confirmation of the Correlation Between High SEMG2 Expression and Infiltration of Regulatory T cells (Treg) with Immunosuppressive Function

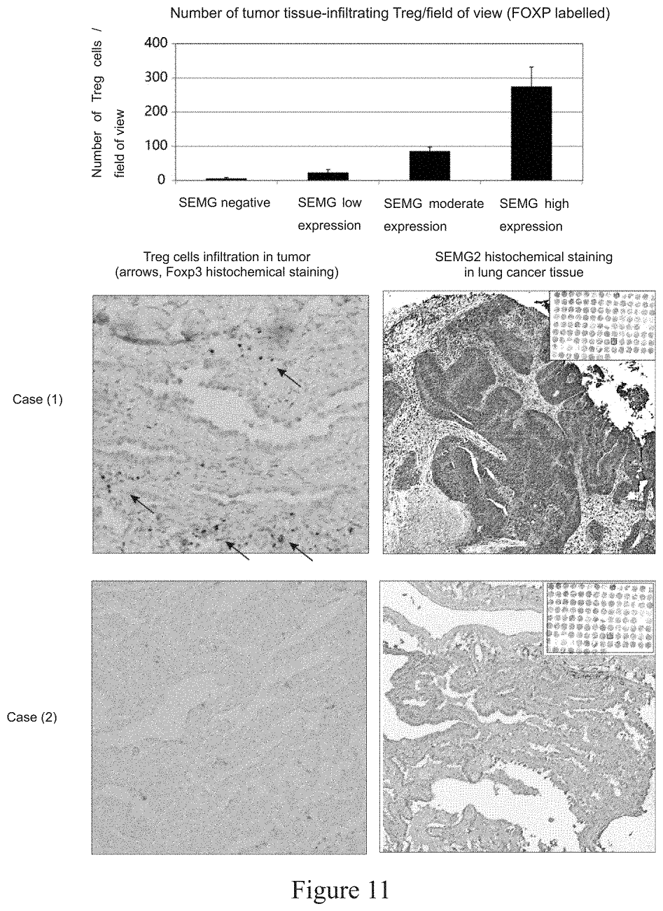

To analyze the correlation between SEMG2 expression in tumor tissues and infiltration of Regulatory T cells (Treg) with immunosuppressive function, we examined a variety of tumor tissue chips purchased from Shanghai Xinchao Biotechnology Co., Ltd using immunohistochemistry assay. Take lung cancer for example, the infiltration of Treg in tumor tissues (labeled by Foxp3 antibody) was compared according to the expression of SEMG2. It was found that the higher the expression of SEMG2 was, the more Treg infiltrated (there was a statistically significant difference among the tissues, P<0.05), see FIG. 11. The result demonstrates that the expression of SEMG2 is significantly correlated with the local immune microenvironment of the tumor, and the expression of SEMG2 can be used as a biomarker of immunosuppression status and a companion diagnostic marker for tumor immunomodulators.

### Example 9: Preparation of Antibody Using SEMG2 (497-509) Fragment as Immunogen

Specifically, the following steps are included: (1) antigen preparation, synthesize polypeptide according to SEMG2 (497-509), i.e., the “QIEKLVEGKSQIQ” sequence, and couple to VLP carrier for immunization; use full-length SEMG2 protein (purchased from Cusabio, Cat. No. CSB-YP0211002HU) as immunogen for another group, (2) The first immunization: remove part of the rabbit hair on both hind paws of the rabbit using a pair of scissors, and disinfect the skin with alcohol and iodine. Aspirate 1 mL of antigen solution emulsify by Freund's complete adjuvant (FCA) using a 2 mL syringe, and inject 0.5 mL of which into each sole of the feet subcutaneously. (3) The second immunization: After an interval of 10-14 days, inject the antigen solution into the swollen lymph nodes on bilateral fossa and groin, 0.1 mL for each lymph node and 1 mL for the rest under the skin near the lymph nodes. If the lymph nodes are not swollen or the swelling is not obvious, directly inject it into bilateral fossa and subcutaneous of groin. (4) After an interval of 7-10 days, collect 0.5-1.0 mL of blood from the ear vein, separate the serum, and determine the serum titer using indirect ELISA which coated with 10 μg/mL of antigen. Collect the blood if the titer is 1:64,000 or more. (5) If the titer does not meet the requirements, inject the antigen liquid without adjuvant into the ear vein for immunization. Which is, inject for 3 times within 1 week, 0.1, 0.3 and 0.5 mL for each time, respectively. Repeat the blood test after an interval of 1 week. If the titer meets the requirement, take the blood immediately, and collect all the antiserum.

The specific experimental steps for polyclonal antibody purification comprise: (1) Preparation of protein A sepharose CL-4B affinity column. To prepare 10 mL of protein A sepharose CL-4B packing, mix equal volume of packing and TBS buffer solution in a vacuum flask, stir and vacuum for 15 minutes to remove air bubbles in the packing. Slowly add Protein A sepharose CL-4B packing into the glass column using the pump to control the filling speed at 1 mL/min-2 mL/min, avoid column dryness, and use 10 times the bed volume of pre-cooled TBS buffer solution to equilibrate the column. (2) Preparation of antiserum. Slowly thaw the antiserum in ice water or in a 4° C. freezer to avoid protein aggregation. Aggregates appeared during protein thawing process can be dissolved by preheating at 37° C. Add solid sodium azide to a concentration of 0.05%, centrifuge at 15,000×g for 5 minutes at 4° C., remove the clarified antiserum and filter through a filter to remove excess lipids. (3) Affinity chromatography. Dilute the antibody with TBS buffer solution at 1:5 and filtered through a filter. Load the antiserum onto the column at a speed of 0.5 mL/min. To ensure the binding of the antiserum to the packing, the column should be loaded continuously for 2 times and the loading effluent should be kept. Wash the column with TBS buffer solution until Aλ280 nm<0.008, add pH 2.7 elution buffer solution, and elute at a speed of 0.5 mL/min until all proteins flow down. Use a 1.5 mL EP tube with 100 μL of neutralizing buffer solution added to collect the eluate in separate tubes. After mixing, check the pH of the eluate with pH test paper. If the pH is lower than 7, use the neutralization buffer to adjust to about pH 7.4 to prevent antibodies denaturation. Add 10 mL of elution buffer solution, pH 1.9, into the column, and collect the eluate until Aλ280 nm<0.008 according to the method. The protein content in each tube was determined using a spectrophotometer.

### Example 10: Blocking Effect Comparison Between SEMG2 (497-509) and Full-Length SEMG2 as Immunogens in Antibody Preparation

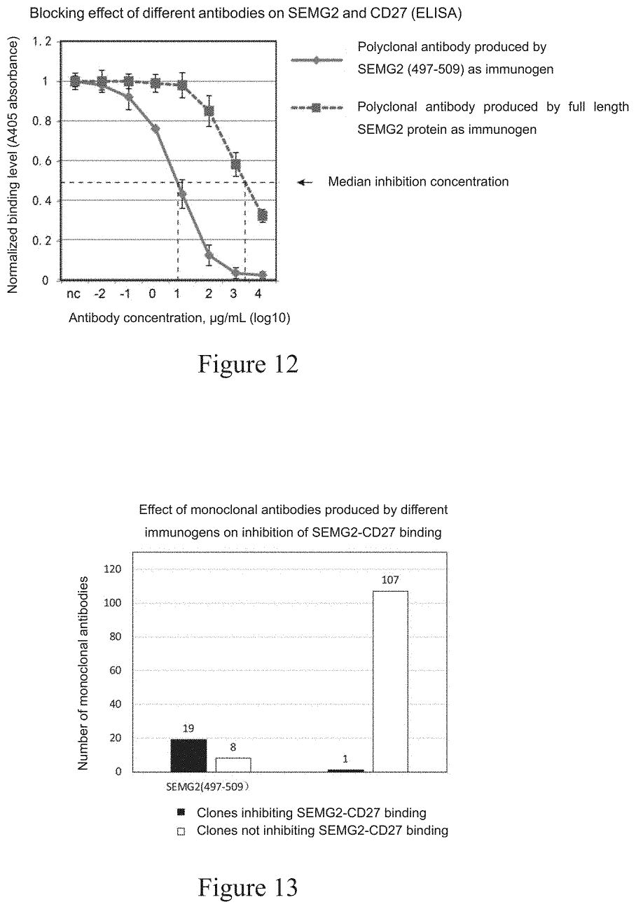

Because SEMG2 (497-509) sequence fragment is the key epitope of SEMG2 binding to CD27, and has a relatively short sequence, so SEMG2 (497-509) was used as an immunogen to prepare antibodies, which is theoretically easier to obtain functional antibody molecules with the function of blocking the binding between SEMG2 and CD27 than using full-length SEMG2 to prepare antibodies. For direct comparison, the differences in effective concentration of producing antibody by the two methods were verified using ELISA in the examples. Antibodies produced with SEMG2 (497-509) as immunogen and antibodies produced with full-length SEMG2 were added into the enzyme-linked immunosorbent assay (ELISA) reaction system at different concentrations (10\hat{ }-2, 10\hat{ }-1, 10\hat{ }0, 10\hat{ }1, 10\hat{ }2, 10\hat{ }3, 10\hat{ }4 μg/mL), and the ELISA binding values were measured. The specific steps of enzyme-linked immunosorbent assay are as follows: (1) Dissolve SEMG2 protein antigen with 50 mM carbonate coating buffer (pH 9.6) to make the antigen concentration 10 μg/mL, and add into 96-well ELISA plate (purchased from Corning) at 100 μL/well, place at 4° C. overnight. (2) After discarding the coating solution on the next day, wash with PBST for three times, add 150 μL of 1% BSA to each well, and block for 2 hours at 37° C. (3) After washing for 3 times with PBST, add the indicated antibodies (polyclonal antibody produced with SEMG2 (497-509) as immunogen, polyclonal antibody produced with full-length SEMG2 as immunogen) to each well to make different final concentrations as shown in FIG. 12, add 10 μg/mL of CD27-Fc fusion protein (i.e., the extracellular region of human CD27 protein fused to the Fe fragment of human antibody), and incubate at 37° C. for 2 hours. (4) After washing for 5 times with PBST, add 100 of diluted HRP-labeled anti-human Fe secondary antibody, and incubate at 37° C. for 1 hour. (5) After washing with PBST for 5 times, 20 min after developing with chromogenic agent, the A450 absorption value was read using the microplate reader.

The experimental results are shown in FIG. 10. The antibody produced by SEMG2 (497-509) as an antigen reduced the binding between SEMG2 and CD27 detected by ELBA by 50% at a lower concentration, while the polyclonal antibody produced by full-length protein of SEMG2 as immunogen only exerted such an effect at higher concentration (the required dose is more than 300 times the former). That is, the blocking titer of the antibody produced by SEMG2 (497-509) was more than 300 times higher than that of the antibody produced by the full-length SEMG2 protein. This indicates that recognition of the key epitope SEMG2 (497-509) makes the development of blocking antibodies easier, and enables those skilled in the art to obtain antibodies that can block the binding between SEMG2 and CD27 more easily.

### Example 11: Preparation of Mouse Monoclonal Antibody Using SEMG2 (497-509) Epitope Peptide and SEMG2 Full-Length Protein

The SEMG2 (497-509) sequence was used to synthesize a polypeptide and coupled to a VLP carrier for immunization; HEK293 cells were used to express the full-length SEMG2 protein, and the purity was tested to reach 92%, and the binding activity of SEMG2 protein to CD27 was verified by ELISA. The protein and polypeptide antigens were used to immunize 10 mice respectively, and multiple immunizations were performed to enhance the effect: (1) The first immunization, 50 μg/mice of antigen, multiple subcutaneous injections together with Freund's complete adjuvant, with an interval of 3 weeks; (2) the second immunization; the same dosage and route as above, with incomplete Freund's adjuvant, and with an interval of 3 weeks; (3) the third immunization, the same dosage as above, without adjuvant, intraperitoneal injection with an interval of 3 weeks; (4) the booster immunization, the dose is 50 μg, intraperitoneal injection. 3 days after the last injection, blood was collected to measure its titer and the immune effect, and mice with higher titers were selected for hybridoma fusion screening. After subcloning, the binding of the monoclonal antibodies to the target antigens was detected by ELISA, and the function of different monoclonal antibodies in blocking the binding between SEMG2 and CD27 was measured by ELISA.

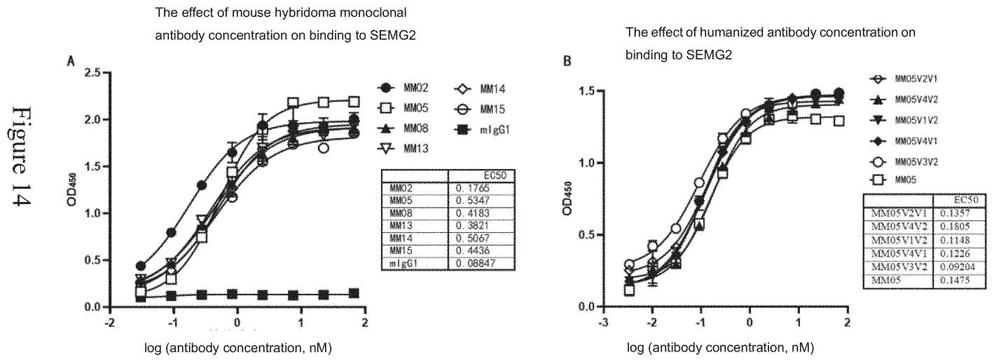

The monoclonal antibodies produced by hybridomas were screened by ELISA. Among the monoclonal antibodies prepared with SEMG2(497-509) as immunogen, 19 strains had blocking function (inhibiting the binding between SEMG2 and CD27) in the first batch of 27 stains of antibodies, as shown in FIG. 13. Among the monoclonal antibodies prepared with the full-length protein of SEMG2 as immunogen, only 1 stain of antibody with blocking function was obtained in a total of 108 stains of antibodies after verification in batches, as shown in FIG. 13.

Therefore, preparing monoclonal antibodies using SEMG2 (497-509) epitope peptide as immunogen significantly improved the efficiency of finding blocking antibodies. The subtypes (Table 2) and sequences (Table 3) of murine monoclonal antibodies are shown in following tables.

TABLE 2
Subtypes of murine monoclonal antibodies

| Antibody clone ID | Subtype | Light chain |
|---|---|---|
| MM02 | mIgG2b | kappa |
| MM05 | mIgG1 | kappa |
| MM07 | mIgG2b | kappa |
| MM08 | mIgG1 | kappa |
| MM13 | mIgG1 | kappa |
| MM14 | mIgG2b | kappa |
| MM15 | mIgG2a | kappa |

TABLE 3
Heavy and light chain variable region sequences of marine monoclonal antibodies
VH amino acid sequences are as follows:

| | |
|---|---|
| MM02 | QIQLVQSGPEVKKPGETVRISCKASGYTLTTAGIQWVQKMPGKGLKWIGWINTHSGVPEYAEDFKGRFAFF |
| | LETSASTAYLQISNLKNEDTATYFCARLGLLGYWGQGTTLTVSS |
| | (SEQ ID NO: 35) |
| MM05 | QVQLQQPGAELVRPGASVKLSCEASGYTFTSYWMNWVKQRPGQGLEWIGMIDPSDSETHYNQMFKDK |
| | ATLTVDKSSSTAYMQLSSLTSEDSAVYYCARYLGGKEGSFDYWGQGTTLTVSS |
| | (SEQ ID NO: 36) |
| MM07 | MDWLWTLLFLMAAAQSIQAQIQLVQSGPELKKPGETVRISCKASGYTLTTAGMQWVQKIPGKGLKWIGW |
| | INTHSGVAEFAEDFKGRFAFSLETSANTAYLQIRNLKNEDTATYFCARLGLLGYWGQGTTLTVSS (SEQ ID |
| | NO: 37) |
| MM08 | QVQLQQPGAELVRPGASVKLSCKSSDYTFTRYWMNWVKQRPGQGLEWIGMIDPSDSETHYNQMFKDK |
| | ATLTVDKSSSTAYMQLSSLTSEDSAVYYCARYLGGKEGSFDYWGQGTTLTVSS |
| | (SEQ ID NO: 38) |
| MM13 | EVQLQQSGAELVRSGASVKLSCTASGFNIKDYYMHWMKQRPEQGLEWIGWIDPENGDNEYAPKFQGKAT |
| | MTADTSSNTAYLQLSSLTSEDTAVYYCNVGGAHYWGQGTTLTVSS |
| | (SEQ ID NO: 39) |
| MM14 | QVQLKESGPGLVAPSQSLSITCTVSGFSLTSYAVSWVRQPPGKGLEWLGIIWGDGSTNYHSALISRLSISKDN |
| | SKSQVFLKLNSLQTDDTATYYCAKQERFSDGYYDGFAYWGQGTLVTVSA |
| | (SEQ ID NO: 40) |
| MM15 | QVQLKESGPGLVAPSQSLSITCTVSGFSLTRYGVSWVRQTPGKGLEWLGIIWGDGSTNYHSALISRLSISKDN |
| | SKSQVFLKLNSLQTDDTATYYCAKQERFSDGYYDGFAYWGQGTLVTVSA |
| | (SEQ ID NO: 41) |

VL amino acid sequences are as follows:

| | |
|---|---|
| MM02 | DILLTQSPAILSVSPGERVSFSCRASQSIGTTIHWYQQRTNGSPRLLIKYASESISGIPSRFSGSGSGTDFTLSI |
| | NSVESEDIADYYCQQSNSWPWTFGGGTKLEIKRA |
| | (SEQ ID NO: 42) |
| MM05 | DIVMSQSPSSLAVSAGEKVTMSCKSSQSLLNSRTRKNYLAWYQQKPGQSPKLLIYWASTRESGVPDRFTGS |
| | GSGTDFTLTISSVQAEDLAVYYCKQSYSLPWTFGGGTKLEIKRA |
| | (SEQ ID NO: 43) |
| MM07 | MVSSAQFLVFLLFWIPASRGDILLTQSPAILSVSPGERVSFSCRASQSIGTTIHWYQQRTNGSPRLLIKYASESI |
| | SGIPSRFSGSGSGTDFTLSINSVESEDIADYYCQQSNSWPWTFGGGTKLEIKRA (SEQ ID NO: 44) |
| MM08 | DIVLTQSPSSLAVSAGERVTMSCKSSQSLFNSRTRKNYLAWYQQKPSQSPKLLLYWASTRESGVPDRFTGSG |
| | SGTDFTLTISSVKTEDLAVYYCKQSYELPWTFGGGTKLEMKRA |
| | (SEQ ID NO: 45) |
| MM13 | DVVMTQTPLSLPVSLGDQASISCRSSQSLVHSNGNTYLHWYLQKPGQSPKLLIYKVSNRFSGVPDRFSSGSGS |
| | GTDFTLKISRVEAEDLGVYFCSQSTHVPYTFGGGTKLEIKRA |
| | (SEQ ID NO: 46) |
| MM14/ | QIVLTQSPAIMSASPGEKVTITCSASSSVSYMHWFQQKPGTSPKLWIYSTSNLASGVPARFSGSGSGTSYSLTI |
| MM15 | SAMEAEDAATYYCQQRSSYPFTFGSGTKLEIKRA |
| | (SEQ ID NO: 47) |

TABLE 4
CDR amino acid sequences of mouse antibodies
VH CDR sequence-IMGT analysis

| Antibody | CDR1 | CDR2 | CDR3 |
|---|---|---|---|
| MM02 | GYTLTTAG | INTHSGVP | ARLGLLGY |
| | (SEQ ID NO: 6) | (SEQ ID NO: 12) | (SEQ ID NO: 17) |
| MM05 | GYTFTSYW | IDPSDSET | ARYLGGKEGSFDY |
| | (SEQ ID NO: 7) | (SEQ ID NO: 13) | (SEQ ID NO: 18) |
| MM07 | GYTLTTAG | INTHSGVA | ARLGLLGY |
| | (SEQ ID NO: 6) | (SEQ ID NO: 16) | (SEQ ID NO: 17) |
| MM08 | DYTFTRYW | IDPSDSET | ARYLGGKEGSFDY |
| | (SEQ IO NO: 8) | (SEQ ID NO: 13) | (SEQ ID NO: 18) |
| MM13 | GFNIKDYY | IDPENGDN | NVGGAHY |
| | (SEQ ID NO: 9) | (SEQ ID NO: 14) | (SEQ ID NO: 19) |
| MM14 | GFSLTSYA | IWGDGST | AKQERFSDGYYDGFAY |
| | (SEQ ID NO: 10) | (SEQ ID NO: 15) | (SEQ ID NO: 20) |
| MM15 | GFSLTRYG | IWGDGST | AKQERFSDGYYDGFAY |
| | (SEQ ID NO: 11) | (SEQ ID NO: 15) | (SEQ ID NO: 20) |

VL CDR sequence-IMGT analysis

| Antibody | CDR1 | CDR2 | CDR3 |
|---|---|---|---|
| MM02 | QSIGTT | YA | QQSNSWPWT |
| | (SEQ ID NO: 21) | (SEQ ID NO: 26) | (SEQ ID NO: 30) |
| MM05 | QSLLNSRTRKNY | WA | KQSYSLPWT |
| | (SEQ ID NO: 22) | (SEQ ID NO: 27) | (SEQ ID NO: 31) |
| MM05-2 | QSLLNSRTRKNY | WA | QQSYSLPWT |
| | (SEQ ID NO: 22) | (SEQ ID NO: 27) | (SEQ ID NO: 95) |
| MM07 | QSIGTT | YA | QQSNSWPWT |
| | (SEQ ID NO: 21) | (SEQ ID NO: 26) | (SEQ ID NO: 30) |
| MM08 | QSLFNSRTRKNY | WA | KQSYELPWT |
| | (SEQ ID NO: 23) | (SEQ ID NO: 27) | (SEQ ID NO: 32) |
| MM13 | QSLVHSNGNTY | KV | SQSTHVPYT |
| | (SEQ ID NO: 24) | (SEQ ID NO: 28) | (SEQ ID NO: 33) |
| MM14 | SSVSY | ST | QQRSSYPFT |
| | (SEQ ID NO: 25) | (SEQ ID NO: 29) | (SEQ ID NO: 34) |
| MM15 | SSVSY | ST | QQRSSYPFT |
| | (SEQ ID NO: 25) | (SEQ ID NO: 29) | (SEQ ID NO: 34) |

The ELISA plate was coated with SEMG2 protein, and the serially diluted murine monoclonal antibody was used as the primary antibody, and the anti-mouse secondary antibody was used to detect the binding abilities of the murine monoclonal antibodies to SEMG2. The results are shown in FIG. 14A. It is shown that the murine monoclonal antibodies have fine affinities for SEMG2 protein.

### Example 12: Humanization of Anti-SEMG2 mAb

The mouse anti-SEMG2 monoclonal antibody MM05 was humanized to reduce immunogenicity when used in human patients. The sequences of the heavy and light chain variable regions (VH and VL) were compared to human antibody sequences in Protein Data Bank (PDB) and homology models were established. The CDRs in the heavy and light chains of mouse mAbs were transplanted to human frame regions that most likely maintain the proper structure required for antigen binding. Reverse mutations or other mutations from human residues to mouse residues were designed when necessary, for example: the amino acid at position 95 of the humanized light chain VL-V2 was mutated from K to Q, and the corresponding CDR3 sequence of the light chain was converted to QQSYSLPWT (SEQ ID NO:95) according to IMGT analysis. Humanized VH and VL regions were fused to the constant regions of heavy chain and K light chain of human IgG1, respectively. Transient transfections were performed in 293E cells using the construction vectors corresponding to mAb sequences, and the binding abilities of the purified mAbs to SEMG2 protein were analyzed using ELISA. Results are shown in absorbance, where higher absorbance indicates a higher level of interaction between the humanized antibody and SEMG2. The amino acid sequences of CDRs, light chain variable regions and heavy chain variable regions, light chains and heavy chains of the 8 humanized antibodies obtained in the present invention are shown in Table 4 and Table 5 below. FIG. 14B shows the fitting curves of the binding of serially diluted humanized monoclonal antibody to SEMG2 protein, and the result show that the humanized antibody maintains the binding ability of murine monoclonal antibody to SEMG⁻2 protein.

TABLE 5
VH and VL amino acid sequences of MM05 humanized antibody
VH amino acid sequences are as follows:

| | |
|---|---|
| VH_V1 | QVQLVQSGAEVKKPGASVKVSCKASGYTFTSYWMNWVRQAPGKGLEWVGMIDPSDSETHYNQMFKD |
| | RVTITADKSTSTAYMELSSLRSEDTAVYYCARYLGGKEGSFDYWGQGTLVTVSS (SEQ ID NO: 48) |
| VH_V2 | QVQLVQSGAEVKKPGASVKVSCKASGYTFTSYWMNWVRQAPGKGLEWVGMIDPSDSETHYNQMFKD |
| | RVTITVDKSTSTAYMELSSLRSEDTAVYYCARYLGGKEGSFDYWGQGTLVTVSS (SEQ ID NO: 49) |
| VH_V3 | QVQLVQSGAEVKKPGASVKVSCKASGYTFTSYWMNWVRQAPGQGLEWVGMIDPSDSETHYAQKFQG |
| | RVTITVDKSTSTVYMELSSLRSEDTAVYYCARYLGGKEGSFDYWGQGTLVTVSS (SEQ ID NO: 50) |
| VH_V4 | QVQLVQSGAEVKKPGASVKVSCKASGYTFTSYWMNWVRQAPGQGLEWVGMIDPSDSETHYAQKFQG |
| | RVTITADKSTSTVYMELSSLRSEDTAVYYCARYLGGKEGSFDYWGQGTLVTVSS (SEQ ID NO: 51) |

VL amino acid sequences are as follows:

| | |
|---|---|
| VL_V1 | DIVMTQSPDSLAVSLGERATINCKSSQSLLNSRTRKNYLAWYQQKPGQPPKLLIYWASTRESGVPDRFSGS |
| | GSGTDFTLTISSLQAEDVAVYYCKQSYSLPWTFGGGTKVEIK (SEQ ID NO: 52) |
| VL_V2 | DIVMTQSPDSLAVSLGERATINCKSSQSLLNSRTRKNYLAWYQQKPGQPPKLLIYWASTRESGVPDRFSGS |
| | GSGTDFTLTISSLQAEDVAVYYCQQSYSLPWTFGGGTKVEIK (SEQ ID NO: 53) |

### Example 13: Functional Comparison Between SEMG2 (497-509) Epitope-Specific Antibodies and Other Epitope-Specific Antibodies in Blocking SEMG2 and CO27 Binding

To demonstrate the importance of the SEMG2 (497-509) epitope for the preparation of blocking antibodies, the functions of antibodies against different SEMG2 epitopes in blocking binding between SEMG2 and CD27 were further compared. It is known that the existing commercial antibodies of HPA042767 and HPA042835 (both purchased from Sigma Aldrich Company) are rabbit polyclonal antibodies against epitopes of SEMG2 (354-403) and SEMG2 (563-574), respectively.

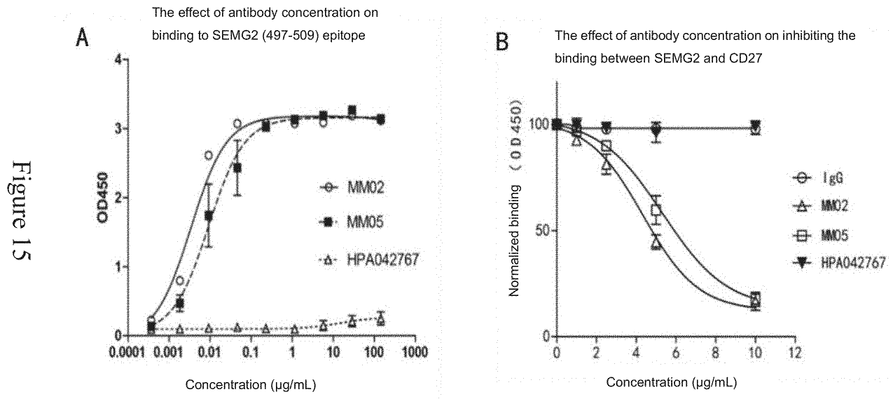

First, SEMG2(497-509) epitope-specific antibodies (for example, MM02, MM05) were compared with other epitope-specific antibodies (for example, HPA042767) for their function in blocking binding between SEMG2 and CD27 at different concentration ranges. The binding of the above antibodies to SEMG2 (497-509) epitope was confirmed by ELISA: MM02 and MM05 were able to hind to SEMG2(497-509), while HPA042767 could not bind to this epitope in a wide range of concentration, as shown in FIG. 15A. The blocking function of different antibodies (irrelative mouse IgG, MM02, MM05, HPA042767) on binding between SEMG2 and CD27 was analyzed by the ELISA experiment as described in Example 11. As shown in FIG. 15B, as the concentrations of MM02 and MM05 increased, the binding of SEMG2 and CD27 gradually decreased, and this phenomenon was observed for both the irrelative mouse IgG and HPA042767 antibodies, indicating that the latter does not possess the function of blocking the binding between SEMG2 and CD27 in a wide range of concentration. These results support the importance of the SEMG2(497-509) epitope in preparing blocking antibodies.

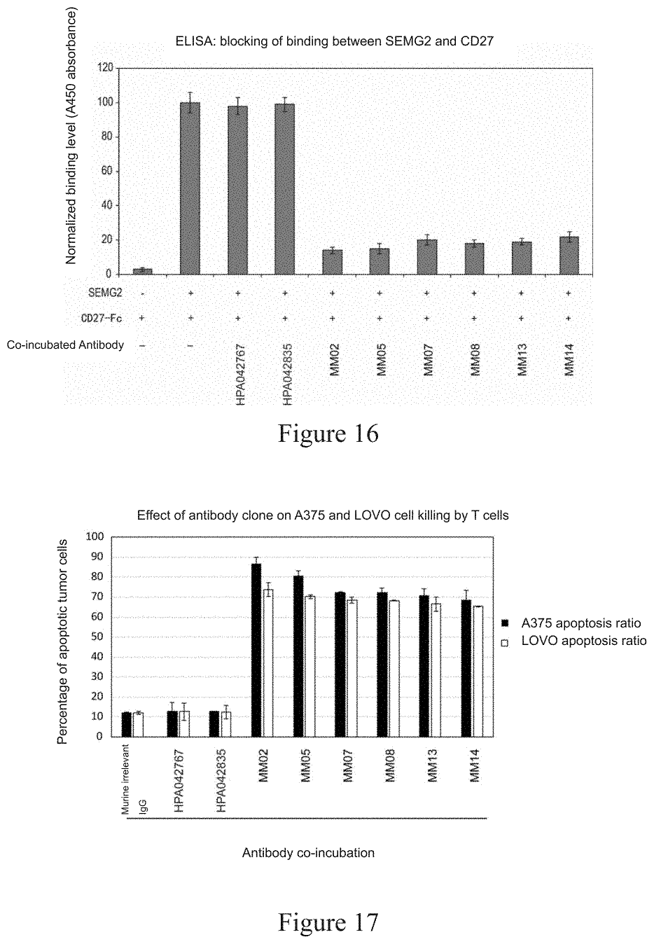

Further, the effects of different antibodies on the binding between SEMG2 and CD27 were compared under the condition of the same antibody concentration. In the ELISA, the same concentration (10 μg/mL) of antibody was used, and the strength of the binding between SEMG2 and CD27 was measured. The results are shown in FIG. 16. MM02, MM05, MM07, MM08, MM13 and MM14 antibodies against SEMG2(497-509) epitope significantly reduced the binding between SEMG2 and CD27; while none of the HPA042767 and HPA042835 antibodies against other epitopes of SEMG2 reduced the binding between SEMG2 and CD27.

### Example 14: Effective Comparison Between SEMG2 (497-509) Epitope-Specific Antibodies and Other Epitope-Specific Antibodies in Tumor Cell Killing by Activated PBMC

In the examples, SEMG2 exerts function of inhibiting activated PBMC from killing tumor cells. Since SEMG2 may play the above role by binding to CD27, and SEMG2 (497-509) epitope is a key site for CD27 binding, SEMG2 (497-509) epitope-specific antibody may neutralize the influence of SEMG2 on tumor cell killing by PBMC.

To test the above hypothesis, the effects of different epitope-specific antibodies on tumor cell killing by activated PBMC were compared. Specifically, A375 human melanoma and LOVO human colorectal cancer cells highly expressing SEMG2 were seeded in 96-well plate. Human peripheral blood mononuclear cells (PBMC; #70025, Stem Cell) were activated with 100 ng/mL of CD3 antibody, 100 ng/mL, of CD28 antibody, and 1.0 ng/mL of IL2 (#317303; #302913; #589102, BioLegend), respectively, and co-cultured with the above tumor cells at a ratio of 10:1 in presence of fluorescent caspase-3/7 substrate (#4440, Essen Bioscience). After 1.0 hours, cells were observed under fluorescence microscope. The results are shown in FIG. 17. Neither HPA042767 nor HPA042835 antibody could affect activated PBMC to kill tumor cells; while MM02, MM05, MM07, MM08, MM13 and MM14 antibodies against SEMG2 (497-509) epitope significantly increased apoptosis tumor cell ratio. The above results indicate that SEMG2 (497-509) epitope-specific antibody can neutralize the activity of SEMG2 (i.e., eliminating the inhibitory effect of SEMG2 on tumor cell killing by PBMC).

### Example 15: Verifying the Correlation Between the Expression Level of SEMG2 and the Promotive Function of Blocking Antibodies in Tumor Cell Killing by PBMC

Since the expression of SEMG2 is a prerequisite for its inhibition of tumor-specific immunity, the expression of SEMG2 is also a potential condition for the suitability of SEMG2-blocking antibody administration. In theory, tumor cells with high SEMG2 expression will have a relative increase in the tumor cell killing by PBMC after neutralizing of SEMG2 activity; tumor cells that do not express SEMG2 may not rely on SEMG2 to play immune escape function, therefore the tumor cell killing by PBMC may not produce a significant change after neutralizing of SEMG2 activity.

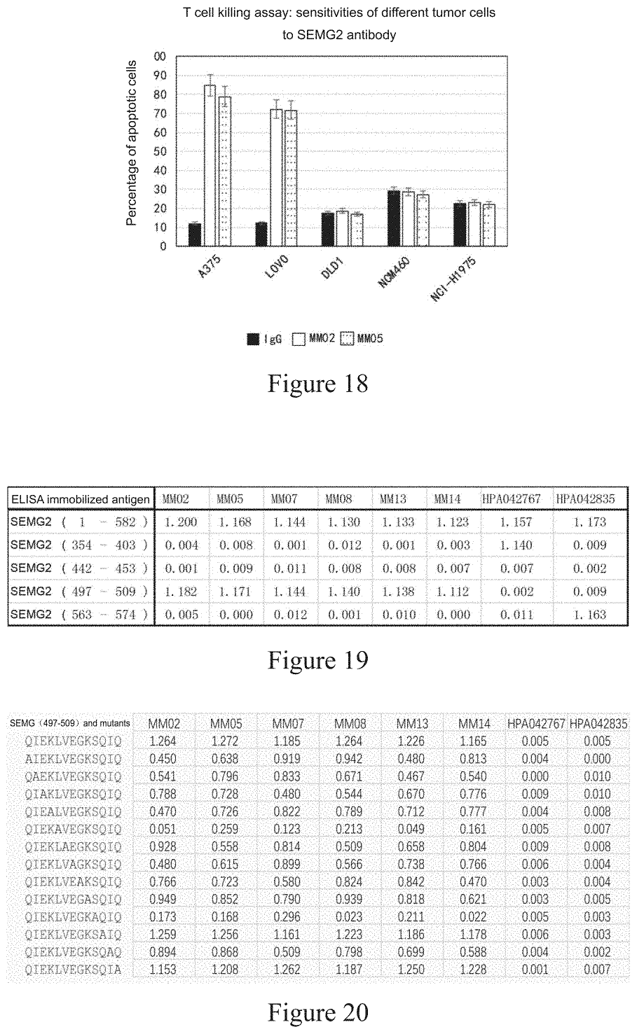

To verify the above hypothesis, tumor cells with high SEMG2 expression (A375, LOVO) and SEMG2-negative tumor cells (DLD1, NCM460 and NCI-H1975) were selected. Different antibodies (irrelevant mouse IgG antibodies, MM02 or MM05 antibodies) were added during the PBMC killing experiments for the tumor cells. Results are shown in FIG. 18. MM02 and MM05 antibodies significantly increased the killing of SEMG2-positive tumor cells (A375, LOVO) by activated PBMC, but has no obvious impact to the killing of SEMG2-negative tumor cells (DLD1, NCM460 and NCI-H1975). Therefore, the above experimental results indicate that the positive expression of SEMG2 is a screening condition for administration of SEMG2 and CD27 blocking antibodies, i.e., a corresponding biomarker.

### Example 16: Accurate Definition of Associated Epitopes of Antibody for Blocking the Binding Between SEMG2 and CD27

To clearly distinguish the binding epitopes of blocking antibodies (i.e., antibodies that can inhibit the binding between SEMG2 and CD27) and non-blocking antibodies, a corresponding ELISA analysis method was established. Specifically, SEMG2 full-length protein (1-582), SEMG2(354-403) fragment, SEMG2(442-453) fragment, SEMG2(497-509) fragment and SEMG2(563-574) fragment were immobilized on the ELISA plate, and the same concentration of antibodies (MM02, MM05, MM07, MM08, MM13, MM14, HPA042767 and HPA042835) were added. Anti-mouse or anti-rabbit secondary antibodies were then used to detect the corresponding bound antibodies. The results are shown in FIG. 19. MM02, MM05, MM07, MM08, MM13, and MM14 all bound to SEMG2(497509) epitope, HPA042767 bound to SEMG2 (354-403) epitope, and HPA042835 bound to SEMG2 (497-509) epitope, while none of the antibodies bound to SEMG2 (442-453) control fragment. These results support the labeling specificity of the antibodies.

To further precisely define the exact epitopes (specific to the level of single amino acid) to which blocking antibodies MM02, MM05, MM07, MM08, MM13, MM14 bind, corresponding ELISA analysis method was established. As shown in FIG. 20, SEMG2(497-509) polypeptide and a group of polypeptide sequences substituted by glycine one by one (glycine mutation scanning sequence group) were immobilized on the ELISA plate, and the same concentration of antibodies (MM02, MM05, MM07, MM08, MM13, MM14, HPA042767 and 1IPA042835) were added respectively. Anti-mouse or anti-rabbit secondary antibodies were then used to detect the corresponding bound antibodies. The results are shown in FIG. 20. The HPA042767 and HPA042835 antibodies did not bind to the sequences, indicating the specificity of the experiment and the different epitope classes of the two types of antibodies. Meanwhile, the different amino acids in the SEMG2(497-509) sequence had different degrees of influence on the binding of similar blocking antibodies (MM02, MM05, MM07, MM08, MM13, MM14) after substitution by glycine. For example: the substitution of amino acids at positions 507 and 509 did not significantly affect the binding of MM02 and similar antibodies; the substitution of amino acids at positions 501 and 506 significantly affected the binding of MM02 and similar antibodies (a decrease of more than 70%); amino acids at other sites affected the binding of MM02 and similar antibodies to a certain extent after substitution by glycine. The results precisely define the epitope amino acids associated with MM02 and similar antibodies (i.e., antibodies that block the binding between SEMG2 and CD27), and the contribution of each amino acid to the binding. In addition, the key amino acids of SEMG2 participating in binding to blocking antibodies are highly consistent with those participating in binding to CD27, which indicates that MM02 and its similar antibodies compete with CD27 for binding to SEMG2, which verifies the molecular mechanism of antibody function.

### Example 17: Preparation and Screening of Fully Human Antibodies Using SEMG2(497-509) Epitope to Block the Binding Between SEMG2 and CD27 and to Promote the Tumor Cell Killing by PBMC

Results of the examples showed the importance of SEMG2(497-509) epitope in the preparation of blocking antibodies, and this epitope was applied to the screening of fully human antibodies. Specifically, the preparation of polypeptide antigens and the screening of human natural antibody library were firstly performed. The SEMG2(497-509) polypeptide was synthesized and coupled to BSA and KLH, respectively, and screened in a fully human phage display antibody library. ELISA was used to select clones that bind to antigenic epitopes for preliminary screening. Different unique sequences were obtained after sequencing single colonies, sorted according to affinity sorting, and full-length antibodies were constructed from antigen-binding fragments (Fab) with relatively high affinity. The binding ability and blocking function tests were performed after purification, that is, the effect of the antibody on binding between SEMG2 and CD27 was determined by the ELISA experiment.

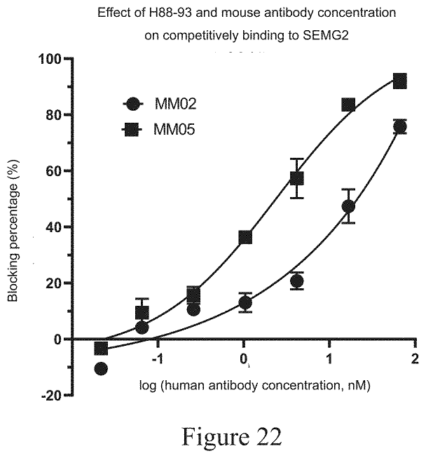

In the same batch of screening, a total of 3 unique sequences of antibodies that bind to SEMG2 (497-509) epitope and inhibit binding of SEMG2 and CD27 were obtained. The three clones were named respectively: H88-93, H88-96 and H88-67, The effect of the corresponding fill-length antibody concentration on binding SEMG2 is shown in FIG. 22A. The amino acid sequences of VH and VL corresponding to the three fully human antibodies and the corresponding CDR sequences are shown in Table 6 and Table 7.

TABLE 6
Variable region sequences of fully human antibodies
VH amino acid sequences are as follows:

| | |
|---|---|
| H88-96 | QVQLLESGGGLVQPGGSLRLSCSASGFTFSSYAMHWVRQAPGKGLEYVSAISSNGGSTYYADSVK |
| | GRFTISRDNSKNTLYLQMSSLRAEDTAVYYCVIEGGSTTGTTSGAFDIWGQGTMVTVSS |
| | (SEQ ID NO: 54) |
| H88-93 | QITLKESGPTLVKPTQTLTLTCNFSGFSLTTSGVGVAWIRQPPGKALEWLALIYWDDDQRYSPSLKSR |
| | LSVTKHTSKDQVVLTMTNVGPVDTATYYCAHLSYGPGWGYYMDVWGNGTMVTVSS |
| | (SEQ ID NO: 55) |
| H88-67 | QVQLLESGGGVVQPGRSLRLSCAASGFTFSSSYAMHWVRQAPGKGLEWVAVISYDGSNKYYADSV |
| | KGRFTISRDNSKNTLYLQMNSLRAEDTAVYYCARMDGSGSPDYWGQGTLVTVSS |
| | (SEQ ID NO: 56) |

VL amino acid sequences are as follows:

| | |
|---|---|
| H88-96 | DIQMIQSPPSVSASVGDTVTIACRANQGIDSWLAWYQQKPGRAPKLLIYSASRLQSGVPSRFSGGG |
| | SGTDFALTISNLQPEDFATYYCQQALSLPITFGQGTRLEIK |
| | (SEQ ID NO: 57) |
| H88-93 | EIVLTQSPGTLSLSPGERASLSCRASQSVRNNYLAWYQQKPGQAPRLLIFGASNRATGIPDTFSGSGS |
| | GTDFTLTISRLEPEDFAVYYCQQYGHSPITFGQGTRLEIK |
| | (SEQ ID NO: 58) |
| H88-67 | DIVMTQSPLSLPVTLGQPASISCRSSQSLVYSDGNTYLNWFQQRPGQSPRRLIYKVSNRDSGVPDRF |
| | SGSGSGTDFTLKISRVEAEDVGVYYCMQGTHWPPAFGQGTKVEIK |
| | (SEQ ID NO: 59) |

TABLE 7
CDR amino acid sequences of human antibodies
VH CDR sequence-IMGT analysis

| Antibody | CDR1 | CDR2 | CDR3 |
|---|---|---|---|
| H88-96 | GFTFSSYA | ISSNGGST | VIEGGSTTGTTSGAFD |
| | (SEQ ID NO: 60) | (SEQ ID NO: 62) | (SEQ ID NO: 65) |
| H88-93 | GFSLTTSGVG | IYWDDDQ | AHLSYGPGWGYYMDV |
| | (SEQ ID NO: 61) | (SEQ ID NO: 63) | (SEQ ID NO: 66) |
| H88-67 | GFTFSSYA | ISYDGSNK | ARMDGSGSPDY |
| | (SEQ ID NO: 60) | (SEQ ID NO: 64) | (SEQ ID NO: 67) |

VL CDR sequence-IMGT analysis

| Antibody | CDR1 | CDR2 | CDR3 |
|---|---|---|---|
| H88-96 | QGIDSW | SA | QQALSLPIT |
| | (SEQ ID NO: 68) | (SEQ ID NO: 71) | (SEQ ID NO: 73) |
| H88-93 | SQSVRNNY | GA | QQYGHSPIT |
| | (SEQ ID NO: 69) | (SEQ ID NO: 72) | (SEQ ID NO: 74) |
| H88-67 | QSLVYSDGNTY | KV | MQGTHWPPA |
| | (SEQ ID NO: 70) | (SEQ ID NO: 28) | (SEQ ID NO: 75) |

The binding abilities of fully human antibodies and murine antibodies to SEMG2 were tested, that is, murine antibodies MM02 and MM05 and concentration gradient diluted filly human antibodies H88-93 were mixed and added as primary antibodies in a 96-well microplate coated with SEMG2. Murine monoclonal antibody bound to SEMG2 was measured using anti-mouse HRP secondary antibody. The blocking percentage is calculated according to the following formula:

Blocking percentage=[1−(A450 of experimental antibody group-Blank control)/(A450 of positive control antibody−A450 of empty control)]×100%

The result shows that H88-93 competes with MM02 and MM05 for binding to SEMG2, as shown in FIG. 22. It shows that fully human antibodies and murine monoclonal antibodies are the same type of antibodies binding to SEMG2, and because MM02, MM05 and H88-93 all bind to the short peptide SEMG2 (497-509), this type of antibodies can be defined as a class of SEMG2 (497-509) binding antibodies.

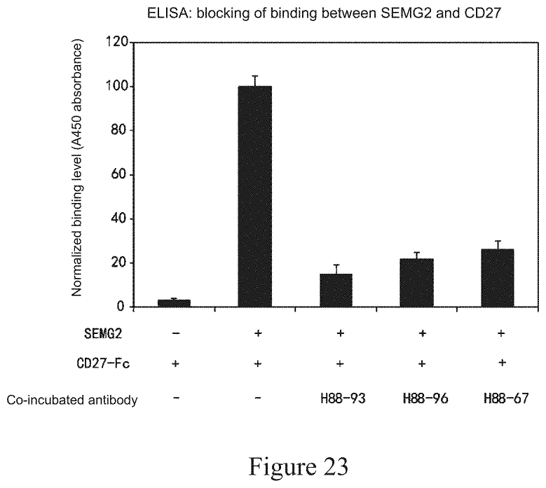

Furthermore, the effect of human antibodies H88-93,H88-96 and H88-67 on blocking the binding between SEMG2 and CD27 was detected by ELISA. All antibodies at a concentration of 10 μg/mL inhibited the binding between SEMG2 and CD27 in varying degrees, as shown in FIG. 23.

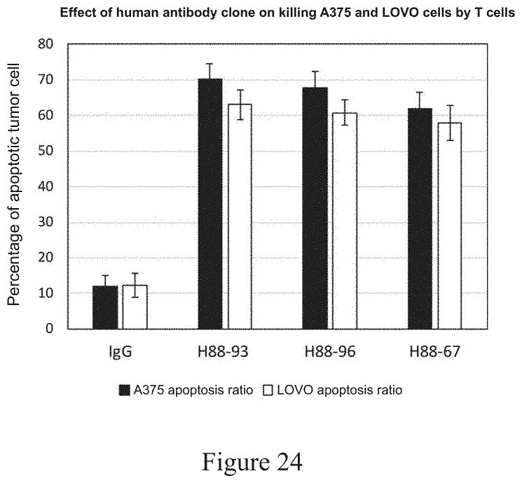

To verify the effect of the human antibodies on the function of activated PBMC on tumor cell killing, A375 and LOVO cells were co-cultured with activated PBMC, and H88-93, H88-96, or H88-67 antibody was added at the same time, and the apoptosis ratio of tumor cells were detected. The result is shown in FIG. 24, which indicates that the three fully human antibodies against SEMG2 (497-509) epitope can significantly promote the killing of SEMG2-expressing tumor cells by PBMC cells.

### Example 17: Binding Kinetic Determination of the Monoclonal Antibodies of the Present Invention to Antigens by Bio-Optical Interferometry

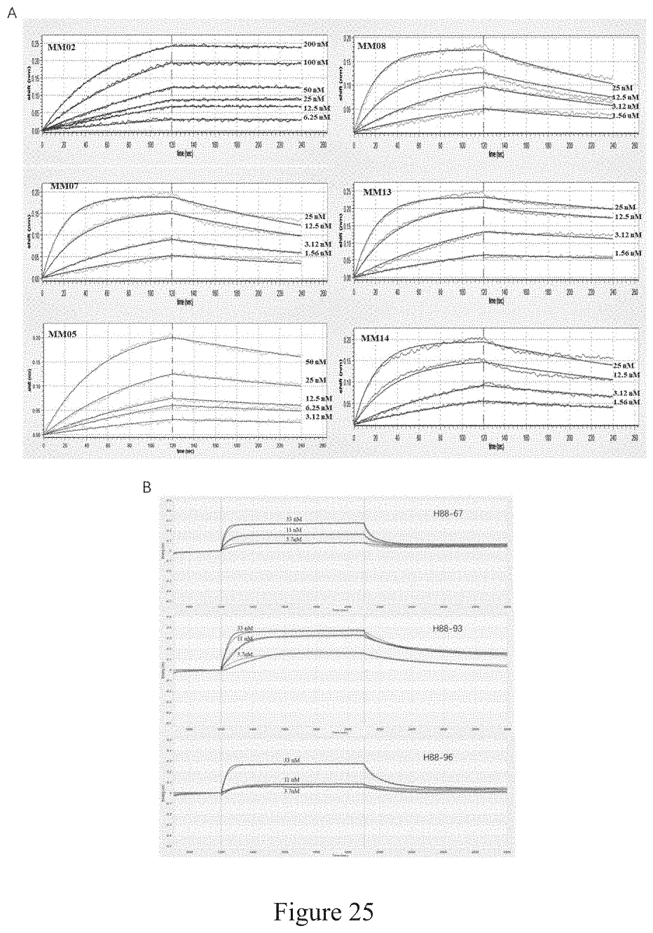

The equilibrium dissociation constant (KD) of the antibody of the present invention binding to human SEMG2 was determined by biolayer interferometry (ForteBio Bltz or Gator instrument). For example, the ForteBio affinity assay was performed according to the existing method, that is, half an hour before start, an appropriate amount of AMQ (Pall, 1506091) (for sample detection) or AHQ (Pall, 1502051) (for positive control detection) sensors were taken and soaked in SD buffer (PBS 1×, BSA 0.1%, Tween-20 0.05%). 100 μl of SD buffer, antibody and SEMG2 were added to a 96-well black polystyrene half area microplate, respectively. Select the sensor location based on the sample location layout. KD values were analyzed using molecular interaction analysis software. In the experiments of the assays, the affinity constants of murine monoclonal antibodies and human antibodies H88-67, H88-93 and H88-96 are shown in Table 8, and the affinity and dissociation curves of SEMG2 and corresponding proteins are shown in FIG. 25.

TABLE 8
Affinity constants (equilibrium dissociation constants) for the detection
of antigen-antibody binding by biolayer optical interferometry

| Antibody | KD (M) |
|---|---|
| MM02 | 1.33 × 10⁻⁹ |
| MM05 | 5.28 × 10⁻⁹ |
| MM07 | 1.82 × 10⁻⁹ |
| MM08 | 2.34 × 10⁻⁹ |
| MM13 | 6.93 × 10⁻¹⁰ |
| MM14 | 1.44 × 10⁻⁹ |
| H88-67 | 2.84 × 10⁻⁸ |
| H88-93 | 4.60 × 10⁻⁹ |
| H88-96 | 1.40 × 10⁻⁸ |

### Example 18: Affinity Maturation of Fully Human Monoclonal Antibodies

Using the plasmids constructed from the VH and VL coding sequences of fully human antibodies H88-96 and H88-67 as templates, the plasmids were obtained by gene synthesis, and then made single-point and double-point saturation mutation. In vitro ligation method was then performed to recombine antibody genes. Finally, the Fab gene sequence of recombinant antibody was inserted into the vector, and then transformed to obtain 4 phage affinity-matured antibody libraries with titer higher than 10⁸CFU. The antibody mutant library was screened by the immunotube gradient screening assay, and the mutants with finely improved affinity compared to the wild type were obtained. The full- length affinity matured human antibody was then constructed according to the detected Fab sequence or the recombination of VH and VL sequences in the Fab sequence. The VH and VL sequences derived from H88-67 and the CDR regions of the VH sequence of H88-96 after affinity maturation are shown in Table 9, and CDR regions of the light and heavy chain of the antibody are shown in Table 9.

TABLE 9
CDR sequences corresponding to affinity matured fully
human antibodies
VH CDR sequence-IMGT analysis

| VH ID | CDR1 | CDR2 | CDR3 |
|---|---|---|---|
| 67-3 | GFTFSSYA | ISYDGSNK | ARMDNHGSPDY |
| | (SEQ ID NO: 60) | (SEQ ID NO: 64) | (SEQ ID NO: 77) |
| 67-6 | GFTFSSYA | ISYDGSNK | ARMDGHGSPDY |
| | (SEQ ID NO: 60) | (SEQ ID NO: 64) | (SEQ ID NO: 78) |
| 67-9 | GFTFSSYA | ISYDGSNK | ARMDSGGSPDY |
| | (SEQ ID NO: 60) | (SEQ ID NO: 64) | (SEQ ID NO: 79) |
| 96-10R | GFTFSSRA | ISSNGGST | VIEGGSTGSTTSGAFDI |
| | (SEQ ID NO: 76) | (SEQ ID NO: 62) | (SEQ ID NO: 80) |
| 96-10V | GFTFSSYA | ISSNGGST | VIEGGSTSSTVSGAFDI |
| | (SEQ ID NO: 60) | (SEQ ID NO: 62) | (SEQ ID NO: 81) |

VL CDR sequence-IMGT analysis

| VL ID | CDR1 | CDR2 | CDR3 |
|---|---|---|---|
| 67-3 | QSLVSDGNTY | KV | MQGTHWPPA |
| | (SEQ ID NO: 70) | (SEQ ID NO: 28) | (SEQ ID NO: 75) |
| 67-4 | QSLVYSDGNTY | EV | MQGTHWPPA |
| | (SEQ ID NO: 70) | (SEQ ID NO: 83) | (SEQ ID NO: 75) |
| 67-5 | QSLVYSDGNTY | GV | MQGTHWPPA |
| | (SEQ ID NO: 70) | (SEQ ID NO: 84) | (SEQ ID NO: 75) |
| 67-6 | QSLVYKDGNTY | KV | MQGTHWPPR |
| | (SEQ ID NO: 82) | (SEQ ID NO: 28) | (SEQ ID NO: 85) |

TABLE 10
VH and VL sequences corresponding to affinity matured fully human antibodies

| Antibody | | |
|---|---|---|
| Number | Heavy chain variable region VH | Light chain variable region VL |
| 67-3-67-3 | QVQLLESGGGVVQPGRSLRLSCAASGFTPS | DIVMTQSPLSLPVTLGQPASISCRSSQSLVYSDGNTYLN |
| | SYAMHWVRQASGKGLEWVAVISYDGSNK | WFQQRPGQSPRRLIYKVSNRDSGVPDRFSGSGSGTDF |
| | YYADSVKGRFTISRDNSKNTLYLQMNSLRA | TLKISRVEAEDVGVYYCMQGTHWPPAFGQGTKVEIK |
| | EDTAVYYCARMDNHGSPDYWGQGTLVTV | (SEQ ID NO: 59) |
| | SS ((SEQ ID NO: 96) | |
| 67-3-67-4 | QVQLLESGGGVVQPGRSLRLSCAASGRTFS | DIVMTQSPLSLPVTLGQPASISCRSSQSLVYSDGNTYLN |
| | SYAMHWVRQASGKGLEWVAVISYDGSNK | WFQQRPGQSPRRLIYEVSNRDSGVPDRFSGSGSGTDF |
| | YYADSVKGRFTISRDNSKNTLYLQMNSLRA | TLKISRVEAEDVGVYYCMQGTHWPPAFGQGTKVEIK |
| | EDTAVYYCARMDNHGSPDYWGQGTLVTV | (SEQ ID NO: 101) |
| | SS ((SEQ ID NO: 96) | |
| 67-3-67-5 | QVQLLESGGGVVQPGRSLRLSCAASGRTFS | DIVMTQSPLSLPVTLGQPASISCRSSQSLVYSDGNTYLN |
| | SYAMHWVRQASGKGLEWVAVISYDGSNK | WFQQRPGQSPRRLIYGVSNRDSGVPDRFSGSGSGTDF |
| | YYADSVKGRFTISRDNSKNTLYLQMNSLRA | TLKISRVEAEDVGVYYCMQGTHWPPAFGQGTKVEIK |
| | EDTAVYYCARMDNHGSPDYWGQGTLVTV | (SEQ ID NO: 102) |
| | SS ((SEQ ID NO: 96) | |
| 67-3-67-6 | QVQLLESGGGVVQPGRSLRLSCAASGRTFS | DIVMTQSPLSLPVTLGQPASISCRSSQSLVYKDGNTYLN |
| | SYAMHWVRQASGKGLEWVAVISYDGSNK | WFQQRPGQSPRRLIYKVSNRDSGVPDRF5G5GSGTDF |
| | YYADSVKGRFTISRDNSKNTLYLQMNSLRA | TLKISRVEAEDVGVYYCMQGTHWPPRFGQGTKVEIK |
| | EDTAVYYCARMDNHGSPDYWGQGTLVTV | (SEQ ID NO: 103) |
| | SS ((SEQ ID NO: 96) | |
| 67-9-67-3 | QVQLLESGGGVVQPGRSLRLSCAASGFTFS | DIVMTQSPLSLPVTLGQPASISCRSSQSLVYSDGNTYLN |
| | SYAMHWVRQASGKGLEWVAVISYDGSNK | WFQQRPGQSPRRLIYKVSNRDSGVPDRFSGSGSGTDF |
| | YYADSVKGRFTISRDNSKNTLYLQMNSLRA | TLKISRVEAEDVGVYYCMQGTHWPPAFGQGTKVEIK |
| | EDTAVYYCARMDSGGSPDYWGQGTLVTV | (SEQ ID NO: 59) |
| | SS ((SEQ ID NO: 97) | |
| 67-6-67-6 | QVQLLESGGGVVQPGRSLRLSCAASGRTFS | DIVMTQSPLSLPVTLGQPASISCRSSQSLVYKDGNTYLN |
| | SYAMHWVRQASGKGLEWVAVISYDGSNK | WFQQRPGQSPRRLIYKVSNRDSGVPDRFSGSGSGTDF |
| | YYADSVKGRFTISRDNSKNTLYLQMNSLRA | TLKISRVEAEDVGVYYCMQGTHWPPRFGQGTKVEIK |
| | EDTAVYYCARMDGHGSPDYWGQGTLVTV | (SEQ ID NO: 103) |
| | SS ((SEQ ID NO: 98) | |
| 96-10R-10 | QVQLLESGGGLVQPGGSLRLSCSASGFTFS | DQMIQSPPSVSASVGDTVTIACRANQGIDSWLAWYQ |
| | SRAMHWVRQAPGKGLEYVSAISSNGGSTY | QKPGRAPKLLYSASRLQSGVPSRFSGGGSGTDFALTIS |
| | YADSVKGRFTISRDNSKNTLYLQMSSLRAE | NLQPEQFATYYCQQALSLPITFGQGTRLEIK |
| | DTAVYYCVIEGGSTGSTTSGAFDIWGQGT | (SEQ ID NO: 57) |
| | MVTVSS ((SEQ ID NO: 99) | |
| 96-10V-10 | QVQLLESGGGLVQPGGSLRLSCSASGFTFS | DIQMQSPPSVSASVGDTVTIACRANQGIDSWLAWYQ |
| | SYAMHWVRQAPGKGLEYVSAISSNGGSTY | QKPGRAPKLLIYSASRLQSGVPSRFSGGGSGTDFALTIS |
| | YADSVKGRFTISRDNSKNTLYLQMSSLRAE | NLQPEDFATYYCQQALSLPITFGQGTRLEIK |
| | DTAVYYCVIEGGSTGSTVSGAFDIWGQGT | (SEQ ID NO: 57) |
| | MVTVSS ((SEQ ID NO: 100) | |

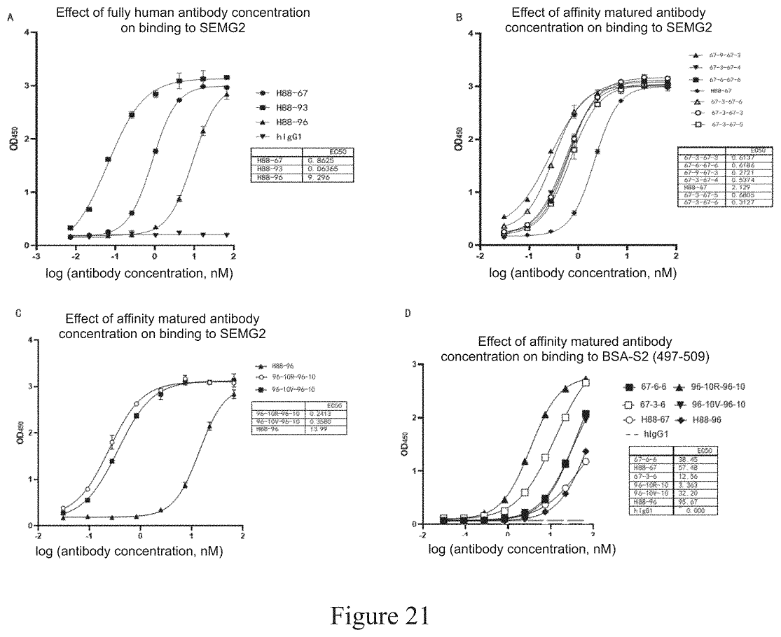

Through the above affinity maturation process, we obtained anti-human SEMG2 monoclonal antibodies with improved affinity, such as 67-3-67-3, 67-3-67-4, 67-3-67-5 and 67-3-67-6 which consist of the combination of the affinity-matured heavy chain numbered 67-3 and the affinity-matured light chain sequence numbered 67-3, 67-4, 67-5 and 67-6, antibody 67-9-67-3 consists of the combination of the heavy chain numbered 67-9 and the light chain numbered 67-3, antibody 67-6-67-6 consists of the combination of the light chain and heavy chain numbered 67-6, and antibodies 96-10R-10 and 96-10V-10 reconstituted by the heavy chains numbered 96-10R and 96-10V and the light chain of H88-96L. The recombinant monoclonal antibodies consist of these light and heavy chains have an affinity more than 10-fold higher for SEMG2 and BSA-S2 (497-509) (i.e., BSA-SP7) than that of the parent antibodies (see FIG. 21B-D).

### Example 19: Validation of the Anti-Tumor Effect of SEMG2 Antibody in Xenograft Model of PBMC-HIS Model in Human Malignant Melanoma A375 Cells

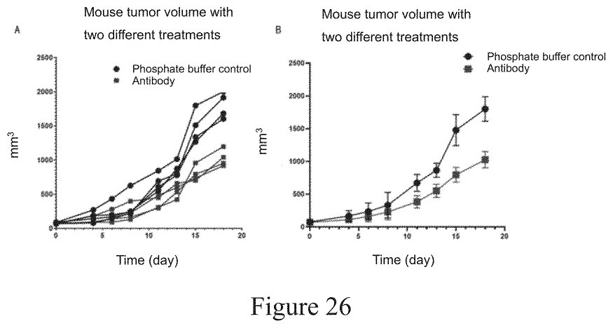

30 male NPSG mouse models aged 6-8 week were weighed. A375 cells (with confirmed endogenous expression of SEMG2) were cultured in vitro to obtain 1.8×10⁸ cells. After 30 mice were inoculated with PBMC, A375 tumor cells were inoculated on the 3rd day. After that, the proportion of hCD45+ cells in mouse blood and the body weight were measured once a week. After inoculation, tumor volume was measured once a week, and the proportion of hCD45+ cells in mouse blood was measured when the average tumor volume reached about 40-80 mm³. Mice were grouped randomly based on tumor volume and the proportion of hCD45+ cells in mouse blood, and the administration was started immediately. The date began the administration was considered day 0. Dosing regimen: SEMG2 antibody (MM05 clone) was injected intraperitoneally at 5 mg/kg three times a week. After the start of administration, the tumor growth status of the mice was observed every week. After the tumor growth, the body weight and tumor volume were measured 3 times a week, and the relative count of hCD45+ cells in mouse blood was monitored by flow cytometry 3 times a week. When the tumor volume reached the end point, blood was collected and the same indexed were detected, and the experiment was ended. The observation of mice includes: daily observation, observation of animal morbidity and death every working day after inoculation. Measurement of tumor volume: after inoculation and before grouping, when tumors were visible, the tumor volume of experimental animals was measured once a week. After inoculation and grouping, the tumor volume of animals in the experiment was measured twice a week. The tumor volume was measured by a bidirectional measurement method. First, the long and short diameters of the tumor were measured with a vernier caliper, and the tumor volume was then calculated using the formula TV=0.5*a*b2, where a is the long diameter of the tumor and b is the short diameter of the tumor. The experimental results are shown in FIG. 26. SEMG2 antibody significantly inhibited the growth of tumor in mice. This result indicates that SEMG2 is an effective anti-tumor target.

### Example 20: Knockout of the Corresponding Gene Svs3a in Mice Proves No Significant Side Effect After Function Blockade of SEMG2

To prove the possible toxic and side effects after functional blockade of SEMG2 as a drug target, the corresponding gene Svs3a in mice was knocked out systemically. The specific scheme was as follows: CRISPR/cas9 technology was adopted in the project, and non-homologous recombination was used to introduce mutation, resulting in a shift in the reading frame and loss of function of Svs3a gene. The brief process is as follows: Cas9 mRNA and gRNA were obtained by in vitro transcription; Cas9 mRNA and gRNA were microinjected into the fertilized eggs of C57BL/6J mice to obtain F0 generation mice. The positive F0 mice verified by PCR amplification and sequencing were mated with C57BL/6J mice to obtain positive F1 mice.

gRNAs sequence (5′-3′):
gRNA1, CAGCCGCAGAGAGGCACTCAGGG;
gRNA2, ATGCACCACCAAGAAACACTGGG.

Sequence alignment before and after knockout:

Wild-type:
TGAGTTCAGGGAGCAGCCGCAGAGAGGCACTCAGGGAGAATGTCCA
TAAGGATGCCATGGCAGTGAGAG......AGTGTCTTAGCAAACGGGAG
AGCTGTCTGCCCCAGTGTTTCTTGGTGGTGCATGGTGGGCTCCCTGT
GCCCGCAGTGC;
Mutant:
TGAGTTCAGGGAGCAGCCGCAAGAGAGG...
(−1006 bp)...GAGGTGCATGGTGGGCTCCCTGTGCCCGCAGTGC.

Subsequent reproduction: the obtained gene knockout heterozygous mice (gene+/−) were divided into two parts: a part of heterozygous mice was mated with wild-type mice for expansion of more heterozygous mice; a part of heterozygous mice self-bred to obtain gene knockout homozygous mice (gene−/−) for gene knockout effect verification and subsequent phenotype analysis.

Phenotype analysis: Anticoagulated whole blood was taken from mice for flow cytometry, and the proportion of CD8+, CD4+, CD3+, CD27+ positive cells in blood was analyzed. After the mice rested for 2 days, the anticoagulated whole blood was collected from the inner canthus, and the molecule department would perform the blood routine test. After the mice rested for 3 days, the mice were weighed and anesthetized, and the mouse gross bodies were imaged; the eyeballs of the mice were removed, the blood was collected, and the serum was separated. The molecule department would measure the serum biochemical parameters. After the eyeball was removed and the blood was collected, the mice were euthanized for material collection: brain: the whole brain was removed and divided by the sagittal plane, and the left side was fixed, and the right side was quick-frozen; liver: the whole liver was removed and divided in two, the left lobe was fixed, and the rest were quick-frozen; spleen: the whole spleen was removed and divided in two, half fixed, half quick-frozen; kidney: the left kidney was removed for fixation, the right kidney was removed and quick-frozen; stomach: the whole stomach was removed and divided sagittal, the greater curvature was fixed, and the lesser curvature was quick-frozen; large intestine: the intact large intestine was removed for Swiss roll fixation; small intestine: the whole small intestine was removed and divided into three sections (duodenum, ileum, jejunum) for Swiss roll fixation; lung: the left lung was removed for fixation, and the right lung for removed for quick freezing; heart: the entire heart was removed for fixation after dilation. All fixed samples were sent to pathology for paraffin embedding, wherein 11 organs (brain, heart, lung, kidney, spleen, liver, stomach, duodenum, jejunum, ileum, and colon) of one KO mouse (#98) were sectioned, HE stained, and read for analysis.

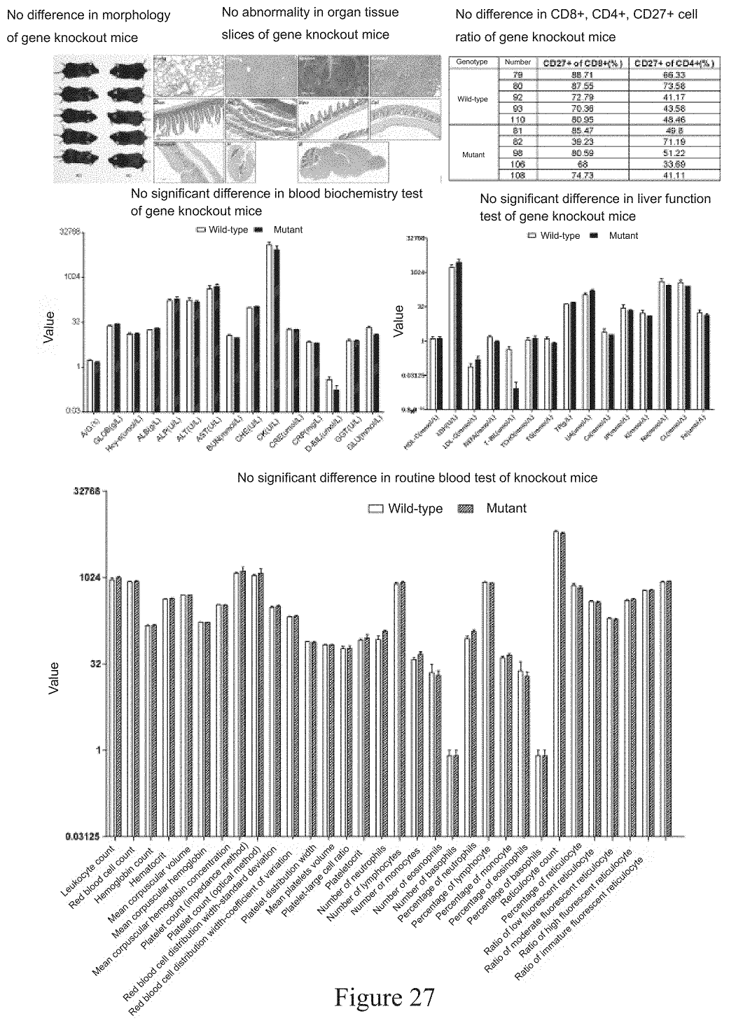

The phenotype analysis results of wild-type (WT) and homozygous knockout mice (KO) are shown in FIG. 27. The results show that no offspring was born after mating the Svs3a homozygous knockout mice, while there was no effect on reproductive function for the heterozygous knockout situation. No abnormality was found in other analyses. Therefore, complete loss or blockade of Svs3a function may affect fertility without significant toxic effects on other systems. This suggests that the possible toxicity or side effects of SEMG2 target blockade are limited and have high safety.

The embodiments of the present invention have been described above by the inventors, but the present invention is not limited thereto, and those skilled in the art can understand that modifications and changes can be made within the scope of the purpose of the present invention. The manner of modifications and changes should fall within the scope of protection of the present invention.

1. A compound agonizing or antagonizing an interaction between SEMG2 and CD27, wherein the interaction between SEMG2 and CD27 is located on the amino acid site at positions 497, 498, 499, 500, 501, 502, 503, 504, 505, 506, and 508 of SEMG2, and the amino acid sequence of the SEMG2 protein is shown in SEQ ID NO:1.

2. (canceled)

3. The compound of claim 1, wherein the compound is a small molecule inhibitor, polypeptide, antibody, or antigen binding fragment;
wherein the polypeptide comprises an amino acid sequence of SEQ ID NO:2 (QIEKLVEGKS), SEQ ID NO:86 (QIEKLVEGKS(x)I(x)), SEQ ID NO:87 (QIEKLVEGKS(x)I), or SEQ ID NO:88 (QIEKLVEGKS(x)); or the polypeptide comprises an amino acid sequence of SEQ ID NO:2, SEQ ID NO:3 (QIEKLVEGKSQIQ), SEQ ID NO:4 (QIEKLVEGKSQ), or SEQ ID NO:5 (QIEKLVEGKSQI), or an amino acid sequence at least 90% identity to an amino acid sequence as provided in SEQ ID NO: 2-5, wherein the x is selected from any amino acid;
wherein the antibody specifically binds to native or mutant SEMG2 protein, the antibody binds to an antigenic epitope peptide derived from SEMG2 protein, the antigenic epitope peptide comprises an amino acid sequence of SEQ ID NO:2 (QIEKLVEGKS), SEQ ID NO:3 (QIEKLVEGKSQIQ), SEQ ID NO:4 (QIEKLVEGKSQ), or SEQ ID NO:5 (QIEKLVEGKSQI); and/or
wherein the antibody specifically binds to native or mutant SEMG2 protein, the antibody recognizes at least one amino acid residue at positions 497, 498, 499, 500, 501, 502, 503, 504, 505, 506 and 508 of the native SEMG2 protein, or recognizes an amino acid residue in the corresponding position of the mutant SEMG2 protein, the amino acid sequence of the native SEMG2 protein is shown in SEQ ID NO:1.

4. (canceled)

5. (canceled)

6. (canceled)

7. The compound of claim 3, wherein the antibody comprises a heavy chain variable region and a light chain variable region, the heavy chain variable region comprises HCDR1, HCDR2 and HCDR3 defined by IMGT; and the light chain variable region comprises LCDR1, LCDR2 and LCDR3 defined by IMGT,
the HCDR1 consists of or comprises an amino acid sequence selected from the group consisting of SEQ ID NOs:6-11, SEQ ID NOs:60-61 and SEQ ID NO:76;
the HCDR2 consists of or comprises an amino acid sequence selected from the group consisting of SEQ ID NOs:12-16 and SEQ ID NOs:62-64;
the HCDR3 consists of or comprises an amino acid sequence selected from the group consisting of SEQ ID NOs:17-20, SEQ ID NOs:65-67 and SEQ ID NOs:77- 81;
the LCDR1 consists of or comprises an amino acid sequence selected from the group consisting of SEQ ID NOs:21-25, SEQ ID NOs:68-70 and SEQ ID NO:82;
the LCDR2 consists of or comprises an amino acid sequence selected from the group consisting of SEQ ID NOs:26-29, SEQ ID NOs:71-72, SEQ ID NOs:83-84 and SEQ ID NO:28;
the LCDR3 consists of or comprises an amino acid sequence selected from the group consisting of SEQ ID NOs:30-34, SEQ ID NOs:73-75, SEQ ID NO:85 and SEQ ID NO:95.

8. The compound of claim 7, wherein the antibody comprises a heavy chain variable region and a light chain variable region, the heavy chain variable region comprises HCDR1, HCDR2 and HCDR3 defined by IMGT; and the light chain variable region comprises LCDR1, LCDR2 and LCDR3 defined by IMGT, the CDR sequence of the antibody is selected from any one of the combinations in (a)-(k):
(a) the HCDR1 comprises the amino acid sequence of SEQ ID NO:6; the HCDR2 comprises the amino acid sequence of SEQ ID NO:12; the HCDR3 comprises the amino acid sequence of SEQ ID NO:17; the LCDR1 comprises the amino acid sequence of SEQ ID NO:21; the LCDR2 comprises the amino acid sequence of SEQ ID NO:26; the LCDR3 comprises the amino acid sequence of SEQ ID NO:30;
(b) the HCDR1 comprises the amino acid sequence of SEQ ID NO:7; the HCDR2 comprises the amino acid sequence of SEQ ID NO:13; the HCDR3 comprises the amino acid sequence of SEQ ID NO:18; the LCDR1 comprises the amino acid sequence of SEQ ID NO:22; the LCDR2 comprises the amino acid sequence of SEQ ID NO:27; the LCDR3 comprises the amino acid sequence of SEQ ID NO:31 or SEQ ID NO:95;
(c) the HCDR1 comprises the amino acid sequence of SEQ ID NO:6; the HCDR2 comprises the amino acid sequence of SEQ ID NO:16; the HCDR3 comprises the amino acid sequence of SEQ ID NO:17; the LCDR1 comprises the amino acid sequence of SEQ ID NO:21; the LCDR2 comprises the amino acid sequence of SEQ ID NO:26; the LCDR3 comprises the amino acid sequence of SEQ ID NO:30;
(d) the HCDR1 comprises the amino acid sequence of SEQ ID NO:8; the HCDR2 comprises the amino acid sequence of SEQ ID NO:13; the HCDR3 comprises the amino acid sequence of SEQ ID NO:18; the LCDR1 comprises the amino acid sequence of SEQ ID NO:23; the LCDR2 comprises the amino acid sequence of SEQ ID NO:27; the LCDR3 comprises the amino acid sequence of SEQ ID NO:32;
(e) the HCDR1 comprises the amino acid sequence of SEQ ID NO:9; the HCDR2 comprises the amino acid sequence of SEQ ID NO:14; the HCDR3 comprises the amino acid sequence of SEQ ID NO:19; the LCDR1 comprises the amino acid sequence of SEQ ID NO:24; the LCDR2 comprises the amino acid sequence of SEQ ID NO:28; the LCDR3 comprises the amino acid sequence of SEQ ID NO:33;
(f) the HCDR1 comprises the amino acid sequence of SEQ ID NO:10; the HCDR2 comprises the amino acid sequence of SEQ ID NO:15; the HCDR3 comprises the amino acid sequence of SEQ ID NO:20; the LCDR1 comprises the amino acid sequence of SEQ ID NO:25; the LCDR2 comprises the amino acid sequence of SEQ ID NO:29; the LCDR3 comprises the amino acid sequence of SEQ ID NO:34;
(g) the HCDR1 comprises the amino acid sequence of SEQ ID NO:11; the HCDR2 comprises the amino acid sequence of SEQ ID NO:15; the HCDR3 comprises the amino acid sequence of SEQ ID NO:20; the LCDR1 comprises the amino acid sequence of SEQ ID NO:25; the LCDR2 comprises the amino acid sequence of SEQ ID NO:29; the LCDR3 comprises the amino acid sequence of SEQ ID NO:34;
(h) the HCDR1 comprises the amino acid sequence of SEQ ID NO:60; the HCDR2 comprises the amino acid sequence of SEQ ID NO:62; the HCDR3 comprises the amino acid sequence of SEQ ID NO:65; the LCDR1 comprises the amino acid sequence of SEQ ID NO:68; the LCDR2 comprises the amino acid sequence of SEQ ID NO:71; the LCDR3 comprises the amino acid sequence of SEQ ID NO:73;
(i) the HCDR1 comprises the amino acid sequence of SEQ ID NO:61; the HCDR2 comprises the amino acid sequence of SEQ ID NO:63; the HCDR3 comprises the amino acid sequence of SEQ ID NO:66; the LCDR1 comprises the amino acid sequence of SEQ ID NO:69; the LCDR2 comprises the amino acid sequence of SEQ ID NO:72; the LCDR3 comprises the amino acid sequence of SEQ ID NO:74;
(j) the HCDR1 comprises the amino acid sequence of SEQ ID NO:60; the HCDR2 comprises the amino acid sequence of SEQ ID NO:64; the HCDR3 comprises the amino acid sequence of SEQ ID NO:67; the LCDR1 comprises the amino acid sequence of SEQ ID NO:70; the LCDR2 comprises the amino acid sequence of SEQ ID NO:28; the LCDR3 comprises the amino acid sequence of SEQ ID NO:75;
(k) the HCDR1 comprises the amino acid sequence of SEQ ID NO:60 or 76; the HCDR2 comprises the amino acid sequence of SEQ ID NO:64 or 62; the HCDR3 comprises the amino acid sequence of SEQ ID NO:77, 78 or 79; and/or
the LCDR1 comprises the amino acid sequence of SEQ ID NO:70 or 82; the LCDR2 comprises the amino acid sequence of SEQ ID NO:28, 83 or 84; the LCDR3 comprises the amino acid sequence of SEQ ID NO:75 or 85.

9. The compound of claim 3, wherein the antibody comprises a heavy chain variable region and a light chain variable region,
the heavy chain variable region comprises an amino acid sequence selected from the group consisting of SEQ ID NOs:35-41, 48-51, 54-56 and 96-100, or an amino acid sequence at least 70%, 80%, 90%, 95% or 99% identity to any sequence of SEQ ID NOs:35-41, 48-51, 54-56 and 96-100;
the light chain variable region comprises an amino acid sequence selected from the group consisting of SEQ ID NOs:42-47, 52-53, 57-69 and 101-103, or an amino acid sequence at least 70%, 80%, 90%, 95% or 99% identity to any sequences of SEQ ID NOs:42-47, 52-53, 57-69 and 101-103.

10. The compound of claim 9, wherein the antibody comprises a heavy chain variable region and a light chain variable region, the heavy chain variable region and the light chain variable region are selected from any one of the combinations in (a)-(o):
(a) the heavy chain variable region comprises SEQ ID NO:35 or an amino acid sequence at least 70%, 80%, 90%, 95% or 99% identity to SEQ ID NO:35; the light chain variable region comprises SEQ ID NO:42 or an amino acid sequence at least 70%, 80%, 90%, 95% or 99% identity to SEQ ID NO:42;
(b) the heavy chain variable region comprises SEQ ID NO:36 or an amino acid sequence at least 70%, 80%, 90%, 95% or 99% identity to SEQ ID NO:36; the light chain variable region comprises SEQ ID NO:43 or an amino acid sequence at least 70%, 80%, 90%, 95% or 99% identity to SEQ ID NO:43;
(c) the heavy chain variable region comprises SEQ ID NO:37 or an amino acid sequence at least 70%, 80%, 90%, 95% or 99% identity to SEQ ID NO:37; the light chain variable region comprises SEQ ID NO:44 or an amino acid sequence at least 70%, 80%, 90%, 95% or 99% identity to SEQ ID NO:44;
(d) the heavy chain variable region comprises SEQ ID NO:38 or an amino acid sequence at least 70%, 80%, 90%, 95% or 99% identity to SEQ ID NO:38; the light chain variable region comprises SEQ ID NO:45 or an amino acid sequence at least 70%, 80%, 90%, 95% or 99% identity to SEQ ID NO:45;
(e) the heavy chain variable region comprises SEQ ID NO:39 or an amino acid sequence at least 70%, 80%, 90%, 95% or 99% identity to SEQ ID NO:39; the light chain variable region comprises SEQ ID NO:46 or an amino acid sequence at least 70%, 80%, 90%, 95% or 99% identity to SEQ ID NO:46;
(f) the heavy chain variable region comprises SEQ ID NO:40 or an amino acid sequence at least 70%, 80%, 90%, 95% or 99% identity to SEQ ID NO:40; the light chain variable region comprises SEQ ID NO:47 or an amino acid sequence at least 70%, 80%, 90%, 95% or 99% identity to SEQ ID NO:47;
(g) the heavy chain variable region comprises SEQ ID NO:41 or an amino acid sequence at least 70%, 80%, 90%, 95% or 99% identity to SEQ ID NO:41; the light chain variable region comprises SEQ ID NO:47 or an amino acid sequence at least 70%, 80%, 90%, 95% or 99% identity to SEQ ID NO:47;
(h) the heavy chain variable region comprises SEQ ID NO:48, 49, 50, 51 or an amino acid sequence at least 70%, 80%, 90%, 95% or 99% identity to SEQ ID NO:48, 49, 50 or 51; the light chain variable region comprises SEQ ID NO:52 or 53 or an amino acid sequence at least 70%, 80%, 90%, 95% or 99% to SEQ ID NO:52 or 53;
(i) the heavy chain variable region comprises SEQ ID NO:54 or an amino acid sequence at least 70%, 80%, 90%, 95% or 99% identity to SEQ ID NO:54; the light chain variable region comprises SEQ ID NO:57 or an amino acid sequence at least 70%, 80%, 90%, 95% or 99% identity to SEQ ID NO:57;
(j) the heavy chain variable region comprises SEQ ID NO:55 or an amino acid sequence at least 70%, 80%, 90%, 95% or 99% identity to SEQ ID NO:55; the light chain variable region comprises SEQ ID NO:58 or an amino acid sequence at least 70%, 80%, 90%, 95% or 99% identity to SEQ ID NO:58;
(k) the heavy chain variable region comprises SEQ ID NO:56 or an amino acid sequence at least 70%, 80%, 90%, 95% or 99% identity to SEQ ID NO:56; the light chain variable region comprises SEQ ID NO:59 or an amino acid sequence at least 70%, 80%, 90%, 95% or 99% identity to SEQ ID NO:59;
(l) the heavy chain variable region comprises SEQ ID NO:96 or an amino acid sequence at least 70%, 80%, 90%, 95% or 99% identity to SEQ ID NO:96; the light chain variable region comprises SEQ ID NO:59, 101, 102, 103 or an amino acid sequence at least 70%, 80%, 90%, 95% or 99% identity to SEQ ID NO:59, 101, 102 or 103;
(m) the heavy chain variable region comprises SEQ ID NO:97 or an amino acid sequence at least 70%, 80%, 90%, 95% or 99% identity to SEQ ID NO:97; the light chain variable region comprises SEQ ID NO:59 or an amino acid sequence at least 70%, 80%, 90%, 95% or 99% identity to SEQ ID NO:59;
(n) the heavy chain variable region comprises SEQ ID NO:98 or an amino acid sequence at least 70%, 80%, 90%, 95% or 99% identity to SEQ ID NO:98; the light chain variable region comprises SEQ ID NO:103 or an amino acid sequence at least 70%, 80%, 90%, 95% or 99% identity to SEQ ID NO:103;
(o) the heavy chain variable region comprises SEQ ID NO:99 or 100 or an amino acid sequence at least 70%, 80%, 90%, 95% or 99% identity to SEQ ID NO:99 or 100; the light chain variable region comprises SEQ ID NO:57 or an amino acid sequence at least 70%, 80%, 90%, 95% or 99% identity to SEQ ID NO:57.

11. The compound of any of the claim 3, wherein the antibody further comprises a coupling moiety linked to the polypeptide, the coupling moiety is selected from the group consisting one or more of radionuclides, drugs, toxins, cytokines, enzymes, fluorescein, carrier proteins, lipids, and biotin, wherein the polypeptide or antibody is selectively linked to the coupling moiety by a linker, preferably the linker is a peptide or polypeptide.

12. The compound of any of the claim 3, wherein the antibody is selected from monoclonal antibodies, polyclonal antibodies, antisera, chimeric antibodies, humanized antibodies, and human antibodies;
wherein the antibody is selected from multispecific antibodies, single chain Fv (scFv), single chain antibodies, anti-idiotype (anti-Id) antibodies, diabodies, minibodies, nanobodies, single domain antibodies, Fab fragments, F(ab′) Fragments, disulfide-linked bispecific Fvs (sdFv) and intracellular antibodies.

13. (canceled)

14. (canceled)

15. A protein, wherein the protein comprises the antigenic epitope peptide of claim 11 and an optional tag sequence which can selectively be linked at the N-terminus or C-terminus;
preferably wherein the protein comprises an amino acid sequence of SEQ ID NO:2 (QIEKLVEGKS), SEQ ID NO:86 (QIEKLVEGKS(x)I(x)), SEQ ID NO:87 (QIEKLVEGKS(x))I), or SEQ ID NO:88 (QIEKLVEGKS(x)), preferably the polypeptide comprises an amino acid sequence of SEQ ID NO:3 (QIEKLVEGKSQIQ), SEQ ID NO:4 (QIEKLVEGKSQ), or SEQ ID NO:5 (QIEKLVEGKSQI) or an amino acid sequence at least 90% identity to any one of SEQ ID NOs:2-5, more preferably SEQ ID NOs:89-94 and SEQ ID NO:3.

16. (canceled)

17. A method for preparing an antibody, including using a protein of claim 15 as immunogen to immunize mammals or obtained by screening in natural antibody library.

18. An isolated polynucleotide encoding the compound of claim 3.

19. A recombinant cloning vector or an expression vector comprising the polynucleotide of claim 18;
wherein the regulatory sequence is selected from a leading sequence, a polyadenylation sequence, a leader-peptide sequence, a promoter, a signal sequence, a transcription terminator, or any combination thereof.

20. (canceled)

21. A host cell comprising the recombinant vector of claim 18;
wherein, the host cell is a prokaryotic cell or a eukaryotic cell.

22. (canceled)

23. A pharmaceutical composition comprising the compound of;
wherein, the pharmaceutical composition further comprises a pharmaceutically acceptable carrier or adjuvant.

24. (canceled)

25. A method for agonizing or antagonizing the interaction between SEMG2 and CD27, comprising administrating the compound of claim 1.

26. (canceled)

27. (canceled)

28. The method of claim 25, wherein; the tumor is selected from one or more of colorectal cancer, lung cancer, melanoma, lymphoma, liver cancer, head and neck cancer, stomach cancer, kidney cancer, bladder cancer, prostate cancer, testicular cancer, endometrial cancer, breast cancer and ovarian cancer.

29. (canceled)

30. (canceled)

31. (canceled)

32. The method of claim 37, wherein the subject has received or is receiving or will receive additional anti-cancer therapy;
wherein the additional anti-cancer therapy comprises surgery, radiotherapy, chemotherapy, immunotherapy, or hormone therapy.

33. (canceled)

34. (canceled)

35. (canceled)

36. The compound for use of claim, wherein the method comprises the following steps: A) analyzing the expression of SEMG2 in tumor cells; B) contacting the tumor cells with an antibody recognizing SEMG2, the binding of the antibody to SEMG2 is KD<2×10⁻⁸; C) contacting T lymphocytes with the antibody and tumor cells

37. A method for preventing or treating tumors, comprising administrating the compound of claim 1 to the subject, wherein SEMG2 is expressed in tumor cells and CD27 is expressed in immune cells.

38. A method for modulating an immune response elicited against tumors, or detecting the presence or absence of SEMG2 in a biological sample in vitro, comprising contacting immune cells such as lymphocytes and/or tumor cells of the subject with an effective dose of the compound of claim 1;
optionally, the expression of SEMG2 in tumor cells is detected before contacting immune cells such as lymphocytes and/or tumor cells of the subject with an effective dose of the compound.


SEQUENCE LISTING (103 sequences)

SEQ ID NO: 1 - protein, 582 aa, Homo sapiens
  UNSURE (1)..(582): Human SEMG2
MKSIILFVLSLLLILEKQAAVMGQKGGSKGQLPSGSSQFPHGQKGQHYFGQKDQQHTKSKGSFSIQHTYHVDINDHDWTRKSQQYDLNALHKATKSKQHLGGSQQLLNYKQEGRDHDKSKGHFHMIVIHHKGGQAHHGTQNPSQDQGNSPSGKGLSSQCSNTEKRLWVHGLSKEQASASGAQKGRTQGGSQSSYVLQTEELVVNKQQRETKNSHQNKGHYQNVVDVREEHSSKLQTSLHPAHQDRLQHGPKDIFTTQDELLVYNKNQHQTKNLSQDQEHGRKAHKISYPSSRTEERQLHHGEKSVQKDVSKGSISIQTEEKIHGKSQNQVTIHSQDQEHGHKENKISYQSSSTEERHLNCGEKGIQKGVSKGSISIQTEEQIHGKSQNQVRIPSQAQEYGHKENKISYQSSSTEERRLNSGEKDVQKGVSKGSISIQTEEKIHGKSQNQVTIPSQDQEHGHKENKMSYQSSSTEERRLNYGGKSTQKDVSQSSISFQIEKLVEGKSQIQTPNPNQDQWSGQNAKGKSGQSADSKQDLLSHEQKGRYKQESSESHNIVITEHEVAQDDHLTQQYNEDRNPIST

SEQ ID NO: 2 - protein, 10 aa, Artificial Sequence
  UNSURE (1)..(10): Epitope peptides
QIEKLVEGKS

SEQ ID NO: 3 - protein, 13 aa, Artificial Sequence
  UNSURE (1)..(13): Epitope peptides
QIEKLVEGKSQIQ

SEQ ID NO: 4 - protein, 11 aa, Artificial Sequence
  UNSURE (1)..(11): Epitope peptides
QIEKLVEGKSQ

SEQ ID NO: 5 - protein, 12 aa, Artificial Sequence
  UNSURE (1)..(12): Epitope peptides
QIEKLVEGKSQI

SEQ ID NO: 6 - protein, 8 aa, Artificial Sequence
  UNSURE (1)..(8): VH CDR1
GYTLTTAG

SEQ ID NO: 7 - protein, 8 aa, Artificial Sequence
  UNSURE (1)..(8): VH CDR1
GYTFTSYW

SEQ ID NO: 8 - protein, 8 aa, Artificial Sequence
  UNSURE (1)..(8): VH CDR1
DYTFTRYW

SEQ ID NO: 9 - protein, 8 aa, Artificial Sequence
  UNSURE (1)..(8): VH CDR1
GFNIKDYY

SEQ ID NO: 10 - protein, 8 aa, Artificial Sequence
  UNSURE (1)..(8): VH CDR1
GFSLTSYA

SEQ ID NO: 11 - protein, 8 aa, Artificial Sequence
  UNSURE (1)..(8): VH CDR1
GFSLTRYG

SEQ ID NO: 12 - protein, 8 aa, Artificial Sequence
  UNSURE (1)..(8): VH CDR2
INTHSGVP

SEQ ID NO: 13 - protein, 8 aa, Artificial Sequence
  UNSURE (1)..(8): VH CDR2
IDPSDSET

SEQ ID NO: 14 - protein, 8 aa, Artificial Sequence
  UNSURE (1)..(8): VH CDR2
IDPENGDN

SEQ ID NO: 15 - protein, 7 aa, Artificial Sequence
  UNSURE (1)..(7): VH CDR2
IWGDGST

SEQ ID NO: 16 - protein, 8 aa, Artificial Sequence
  UNSURE (1)..(8): VH CDR2
INTHSGVA

SEQ ID NO: 17 - protein, 8 aa, Artificial Sequence
  UNSURE (1)..(8): VH CDR3
ARLGLLGY

SEQ ID NO: 18 - protein, 13 aa, Artificial Sequence
  UNSURE (1)..(13): VH CDR3
ARYLGGKEGSFDY

SEQ ID NO: 19 - protein, 7 aa, Artificial Sequence
  UNSURE (1)..(7): VH CDR3
NVGGAHY

SEQ ID NO: 20 - protein, 16 aa, Artificial Sequence
  UNSURE (1)..(16): VH CDR3
AKQERFSDGYYDGFAY

SEQ ID NO: 21 - protein, 6 aa, Artificial Sequence
  UNSURE (1)..(6): VL CDR1
QSIGTT

SEQ ID NO: 22 - protein, 12 aa, Artificial Sequence
  UNSURE (1)..(12): VL CDR1
QSLLNSRTRKNY

SEQ ID NO: 23 - protein, 12 aa, Artificial Sequence
  UNSURE (1)..(12): VL CDR1
QSLFNSRTRKNY

SEQ ID NO: 24 - protein, 11 aa, Artificial Sequence
  UNSURE (1)..(11): VL CDR1
QSLVHSNGNTY

SEQ ID NO: 25 - protein, 5 aa, Artificial Sequence
  UNSURE (1)..(5): VL CDR1
SSVSY

SEQ ID NO: 26 - protein, 2 aa, Artificial Sequence
  UNSURE (1)..(2): VL CDR2
YA

SEQ ID NO: 27 - protein, 2 aa, Artificial Sequence
  UNSURE (1)..(2): VL CDR2
WA

SEQ ID NO: 28 - protein, 2 aa, Artificial Sequence
  UNSURE (1)..(2): VL CDR2
KV

SEQ ID NO: 29 - protein, 2 aa, Artificial Sequence
  UNSURE (1)..(2): VL CDR2
ST

SEQ ID NO: 30 - protein, 9 aa, Artificial Sequence
  UNSURE (1)..(9): VL CDR3
QQSNSWPWT

SEQ ID NO: 31 - protein, 9 aa, Artificial Sequence
  UNSURE (1)..(9): VL CDR3
KQSYSLPWT

SEQ ID NO: 32 - protein, 9 aa, Artificial Sequence
  UNSURE (1)..(9): VL CDR3
KQSYELPWT

SEQ ID NO: 33 - protein, 9 aa, Artificial Sequence
  UNSURE (1)..(9): VL CDR3
SQSTHVPYT

SEQ ID NO: 34 - protein, 9 aa, Artificial Sequence
  UNSURE (1)..(9): VL CDR3
QQRSSYPFT

SEQ ID NO: 35 - protein, 115 aa, Artificial Sequence
  UNSURE (1)..(115): VH
QIQLVQSGPEVKKPGETVRISCKASGYTLTTAGIQWVQKMPGKGLKWIGWINTHSGVPEYAEDFKGRFAFFLETSASTAYLQISNLKNEDTATYFCARLGLLGYWGQGTTLTVSS

SEQ ID NO: 36 - protein, 120 aa, Artificial Sequence
  UNSURE (1)..(120): VH
QVQLQQPGAELVRPGASVKLSCEASGYTFTSYWMNWVKQRPGQGLEWIGMIDPSDSETHYNQMFKDKATLTVDKSSSTAYMQLSSLTSEDSAVYYCARYLGGKEGSFDYWGQGTTLTVSS

SEQ ID NO: 37 - protein, 134 aa, Artificial Sequence
  UNSURE (1)..(134): VH
MDWLWTLLFLMAAAQSIQAQIQLVQSGPELKKPGETVRISCKASGYTLTTAGMQWVQKIPGKGLKWIGWINTHSGVAEFAEDFKGRFAFSLETSANTAYLQIRNLKNEDTATYFCARLGLLGYWGQGTTLTVSS

SEQ ID NO: 38 - protein, 120 aa, Artificial Sequence
  UNSURE (1)..(120): VH
QVQLQQPGAELVRPGASVKLSCKSSDYTFTRYWMNWVKQRPGQGLEWIGMIDPSDSETHHNQMFKDKATLTVDKSSSTAYMQLSSLTSEDSAVYFCARYLGGKEGSFDYWGQGTTLTVSS

SEQ ID NO: 39 - protein, 114 aa, Artificial Sequence
  UNSURE (1)..(114): VH
EVQLQQSGAELVRSGASVKLSCTASGFNIKDYYMHWMKQRPEQGLEWIGWIDPENGDNEYAPKFQGKATMTADTSSNTAYLQLSSLTSEDTAVYYCNVGGAHYWGQGTTLTVSS

SEQ ID NO: 40 - protein, 122 aa, Artificial Sequence
  UNSURE (1)..(122): VH
QVQLKESGPGLVAPSQSLSITCTVSGFSLTSYAVSWVRQPPGKGLEWLGIIWGDGSTNYHSALISRLSISKDNSKSQVFLKLNSLQTDDTATYYCAKQERFSDGYYDGFAYWGQGTLVTVSA

SEQ ID NO: 41 - protein, 122 aa, Artificial Sequence
  UNSURE (1)..(122): VH
QVQLKESGPGLVAPSQSLSITCTVSGFSLTRYGVSWVRQTPGKGLEWLGIIWGDGSTNYHSALISRLSISKDNSKSQVFLKLNSLQTDDTATYYCAKQERFSDGYYDGFAYWGQGTLVTVSA

SEQ ID NO: 42 - protein, 109 aa, Artificial Sequence
  UNSURE (1)..(109): VL
DILLTQSPAILSVSPGERVSFSCRASQSIGTTIHWYQQRTNGSPRLLIKYASESISGIPSRFSGSGSGTDFTLSINSVESEDIADYYCQQSNSWPWTFGGGTKLEIKRA

SEQ ID NO: 43 - protein, 115 aa, Artificial Sequence
  UNSURE (1)..(115): VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYSLPWTFGGGTKLEIKRA

SEQ ID NO: 44 - protein, 129 aa, Artificial Sequence
  UNSURE (1)..(129): VL
MVSSAQFLVFLLFWIPASRGDILLTQSPAILSVSPGERVSFSCRASQSIGTTIHWYQQRTNGSPRLLIKYASESISGIPSRFSGSGSGTDFTLSINSVESEDIADYYCQQSNSWPWTFGGGTKLEIKRA

SEQ ID NO: 45 - protein, 115 aa, Artificial Sequence
  UNSURE (1)..(115): VL
DIVLTQSPSSLAVSAGERVTMSCKSSQSLFNSRTRKNYLAWYQQKPGQSPKLLLYWASTRESGVPDRFTGSGSGTDFTLTISSVKTEDLAVYYCKQSYELPWTFGGGTKLEMKRA

SEQ ID NO: 46 - protein, 114 aa, Artificial Sequence
  UNSURE (1)..(114): VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKRA

SEQ ID NO: 47 - protein, 108 aa, Artificial Sequence
  UNSURE (1)..(108): VL
QIVLTQSPAIMSASPGEKVTITCSASSSVSYMHWFQQKPGTSPKLWIYSTSNLASGVPARFSGSGSGTSYSLTISRMEAEDAATYYCQQRSSYPFTFGSGTKLEIKRA

SEQ ID NO: 48 - protein, 120 aa, Artificial Sequence
  UNSURE (1)..(120): VH
QVQLVQSGAEVKKPGASVKVSCKASGYTFTSYWMNWVRQAPGKGLEWVGMIDPSDSETHYNQMFKDRVTITADKSTSTAYMELSSLRSEDTAVYYCARYLGGKEGSFDYWGQGTLVTVSS

SEQ ID NO: 49 - protein, 120 aa, Artificial Sequence
  UNSURE (1)..(120): VH
QVQLVQSGAEVKKPGASVKVSCKASGYTFTSYWMNWVRQAPGKGLEWVGMIDPSDSETHYNQMFKDRVTITVDKSTSTAYMELSSLRSEDTAVYYCARYLGGKEGSFDYWGQGTLVTVSS

SEQ ID NO: 50 - protein, 120 aa, Artificial Sequence
  UNSURE (1)..(120): VH
QVQLVQSGAEVKKPGASVKVSCKASGYTFTSYWMNWVRQAPGQGLEWVGMIDPSDSETHYAQKFQGRVTITVDKSTSTVYMELSSLRSEDTAVYYCARYLGGKEGSFDYWGQGTLVTVSS

SEQ ID NO: 51 - protein, 120 aa, Artificial Sequence
  UNSURE (1)..(120): VH
QVQLVQSGAEVKKPGASVKVSCKASGYTFTSYWMNWVRQAPGQGLEWVGMIDPSDSETHYAQKFQGRVTITADKSTSTVYMELSSLRSEDTAVYYCARYLGGKEGSFDYWGQGTLVTVSS

SEQ ID NO: 52 - protein, 113 aa, Artificial Sequence
  UNSURE (1)..(113): VL
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYSLPWTFGGGTKVEIK

SEQ ID NO: 53 - protein, 113 aa, Artificial Sequence
  UNSURE (1)..(113): VL
DIVMTQSPDSLAVSLGERATINCKSSQSLLNSRTRKNYLAWYQQKPGQPPKLLIYWASTRESGVPDRFSGSGSGTDFTLTISSLQAEDVAVYYCQQSYSLPWTFGGGTKVEIK

SEQ ID NO: 54 - protein, 124 aa, Artificial Sequence
  UNSURE (1)..(124): VH
QVQLLESGGGLVQPGGSLRLSCSASGFTFSSYAMHWVRQAPGKGLEYVSAISSNGGSTYYADSVKGRFTISRDNSKNTLYLQMSSLRAEDTAVYYCVIEGGSTTGTTSGAFDIWGQGTMVTVSS

SEQ ID NO: 55 - protein, 123 aa, Artificial Sequence
  UNSURE (1)..(123): VH
QITLKESGPTLVKPTQTLTLTCNFSGFSLTTSGVGVAWIRQPPGKALEWLALIYWDDDQRYSPSLKSRLSVTKHTSKDQVVLTMTNVGPVDTATYYCAHLSYGPGWGYYMDVWGNGTMVTVSS

SEQ ID NO: 56 - protein, 118 aa, Artificial Sequence
  UNSURE (1)..(118): VH
QVQLLESGGGVVQPGRSLRLSCAASGFTFSSYAMHWVRQAPGKGLEWVAVISYDGSNKYYADSVKGRFTISRDNSKNTLYLQMNSLRAEDTAVYYCARMDGSGSPDYWGQGTLVTVSS

SEQ ID NO: 57 - protein, 107 aa, Artificial Sequence
  UNSURE (1)..(107): VL
DIQMIQSPPSVSASVGDTVTIACRANQGIDSWLAWYQQKPGRAPKLLIYSASRLQSGVPSRFSGGGSGTDFALTISNLQPEDFATYYCQQALSLPITFGQGTRLEIK

SEQ ID NO: 58 - protein, 108 aa, Artificial Sequence
  UNSURE (1)..(108): VL
EIVLTQSPGTLSLSPGERASLSCRASQSVRNNYLAWYQQKPGQAPRLLIFGASNRATGIPDTFSGSGSGTDFTLTISRLEPEDFAVYYCQQYGHSPITFGQGTRLEIK

SEQ ID NO: 59 - protein, 112 aa, Artificial Sequence
  UNSURE (1)..(112): VL
DIVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPPAFGQGTKVEIK

SEQ ID NO: 60 - protein, 8 aa, Artificial Sequence
  UNSURE (1)..(8): VH CDR1
GFTFSSYA

SEQ ID NO: 61 - protein, 10 aa, Artificial Sequence
  UNSURE (1)..(10): VH CDR1
GFSLTTSGVG

SEQ ID NO: 62 - protein, 8 aa, Artificial Sequence
  UNSURE (1)..(8): VH CDR2
ISSNGGST

SEQ ID NO: 63 - protein, 7 aa, Artificial Sequence
  UNSURE (1)..(7): VH CDR2
IYWDDDQ

SEQ ID NO: 64 - protein, 8 aa, Artificial Sequence
  UNSURE (1)..(8): VH CDR2
ISYDGSNK

SEQ ID NO: 65 - protein, 16 aa, Artificial Sequence
  UNSURE (1)..(16): VH CDR3
VIEGGSTTGTTSGAFD

SEQ ID NO: 66 - protein, 15 aa, Artificial Sequence
  UNSURE (1)..(15): VH CDR3
AHLSYGPGWGYYMDV

SEQ ID NO: 67 - protein, 11 aa, Artificial Sequence
  UNSURE (1)..(11): VH CDR3
ARMDGSGSPDY

SEQ ID NO: 68 - protein, 6 aa, Artificial Sequence
  UNSURE (1)..(6): VL CDR1
QGIDSW

SEQ ID NO: 69 - protein, 8 aa, Artificial Sequence
  UNSURE (1)..(8): VL CDR1
SQSVRNNY

SEQ ID NO: 70 - protein, 11 aa, Artificial Sequence
  UNSURE (1)..(11): VL CDR1
QSLVYSDGNTY

SEQ ID NO: 71 - protein, 2 aa, Artificial Sequence
  UNSURE (1)..(2): VL CDR2
SA

SEQ ID NO: 72 - protein, 2 aa, Artificial Sequence
  UNSURE (1)..(2): VL CDR2
GA

SEQ ID NO: 73 - protein, 9 aa, Artificial Sequence
  UNSURE (1)..(9): VL CDR3
QQALSLPIT

SEQ ID NO: 74 - protein, 9 aa, Artificial Sequence
  UNSURE (1)..(9): VL CDR3
QQYGHSPIT

SEQ ID NO: 75 - protein, 9 aa, Artificial Sequence
  UNSURE (1)..(9): VL CDR3
MQGTHWPPA

SEQ ID NO: 76 - protein, 8 aa, Artificial Sequence
  UNSURE (1)..(8): VH CDR1
GFTFSSRA

SEQ ID NO: 77 - protein, 11 aa, Artificial Sequence
  UNSURE (1)..(11): VH CDR3
ARMDNHGSPDY

SEQ ID NO: 78 - protein, 11 aa, Artificial Sequence
  UNSURE (1)..(11): VH CDR3
ARMDGHGSPDY

SEQ ID NO: 79 - protein, 11 aa, Artificial Sequence
  UNSURE (1)..(11): VH CDR3
ARMDSGGSPDY

SEQ ID NO: 80 - protein, 17 aa, Artificial Sequence
  UNSURE (1)..(17): VH CDR3
VIEGGSTGSTTSGAFDI

SEQ ID NO: 81 - protein, 17 aa, Artificial Sequence
  UNSURE (1)..(17): VH CDR3
VIEGGSTGSTVSGAFDI

SEQ ID NO: 82 - protein, 11 aa, Artificial Sequence
  UNSURE (1)..(11): VL CDR1
QSLVYKDGNTY

SEQ ID NO: 83 - protein, 2 aa, Artificial Sequence
  UNSURE (1)..(2): VL CDR2
EV

SEQ ID NO: 84 - protein, 2 aa, Artificial Sequence
  UNSURE (1)..(2): VL CDR2
GV

SEQ ID NO: 85 - protein, 9 aa, Artificial Sequence
  UNSURE (1)..(9): VL CDR3
MQGTHWPPR

SEQ ID NO: 86 - protein, 13 aa, Artificial Sequence
  UNSURE (1)..(13): Peptide
  UNSURE (11)..(11): Arbitrary amino acid
  UNSURE (13)..(13): Arbitrary amino acid
QIEKLVEGKSXIX

SEQ ID NO: 87 - protein, 12 aa, Artificial Sequence
  UNSURE (1)..(12): Peptide
  UNSURE (11)..(11): Arbitrary amino acid
QIEKLVEGKSXI

SEQ ID NO: 88 - protein, 11 aa, Artificial Sequence
  UNSURE (1)..(11): Peptide
  UNSURE (11)..(11): Arbitrary amino acid
QIEKLVEGKSX

SEQ ID NO: 89 - protein, 79 aa, Artificial Sequence
  UNSURE (1)..(79): SEMG2-P1
GSFSIQHTYHVDINDHDWTRKSQQYDLNALHKATKSKQHLGGSQQLLNYKQEGRDHDKSKGHFHMIVIHHKGGQAHHGT

SEQ ID NO: 90 - protein, 142 aa, Artificial Sequence
  UNSURE (1)..(142): SEMG2-P2
QNPSQDQGNSPSGKGLSSQCSNTEKRLWVHGLSKEQASASGAQKGRTQGGSQSSYVLQTEELVVNKQQRETKNSHQNKGHYQNVVDVREEHSSKLQTSLHPAHQDRLQHGPKDIFTTQDELLVYNKNQHQTKNLSQDQEHGR

SEQ ID NO: 91 - protein, 116 aa, Artificial Sequence
  UNSURE (1)..(116): SEMG2-P3
KAHKISYPSSRTEERQLHHGEKSVQKDVSKGSISIQTEEKIHGKSQNQVTIHSQDQEHGHKENKISYQSSSTEERHLNCGEKGIQKGVSKGSISIQTEEQIHGKSQNQVRIPSQAQ

SEQ ID NO: 92 - protein, 98 aa, Artificial Sequence
  UNSURE (1)..(98): SEMG2-P4
EYGHKENKISYQSSSTEERRLNSGEKDVQKGVSKGSISIQTEEKIHGKSQNQVTIPSQDQEHGHKENKMSYQSSSTEERRLNYGGKSTQKDVSQSSIS

SEQ ID NO: 93 - protein, 45 aa, Artificial Sequence
  UNSURE (1)..(45): SEMG2-P5
FQIEKLVEGKSQIQTPNPNQDQWSGQNAKGKSGQSADSKQDLLSH

SEQ ID NO: 94 - protein, 42 aa, Artificial Sequence
  UNSURE (1)..(42): SEMG2-P6
EQKGRYKQESSESHNIVITEHEVAQDDHLTQQYNEDRNPIST

SEQ ID NO: 95 - protein, 9 aa, Artificial Sequence
  UNSURE (1)..(9): VL CDR3
QQSYSLPWT

SEQ ID NO: 96 - protein, 118 aa, Artificial Sequence
  UNSURE (1)..(118): VH
QVQLLESGGGVVQPGRSLRLSCAASGFTFSSYAMHWVRQASGKGLEWVAVISYDGSNKYYADSVKGRFTISRDNSKNTLYLQMNSLRAEDTAVYYCARMDNHGSPDYWGQGTLVTVSS

SEQ ID NO: 97 - protein, 118 aa, Artificial Sequence
  UNSURE (1)..(118): VH
QVQLLESGGGVVQPGRSLRLSCAASGFTFSSYAMHWVRQASGKGLEWVAVISYDGSNKYYADSVKGRFTISRDNSKNTLYLQMNSLRAEDTAVYYCARMDSGGSPDYWGQGTLVTVSS

SEQ ID NO: 98 - protein, 118 aa, Artificial Sequence
  UNSURE (1)..(118): VH
QVQLLESGGGVVQPGRSLRLSCAASGFTFSSYAMHWVRQASGKGLEWVAVISYDGSNKYYADSVKGRFTISRDNSKNTLYLQMNSLRAEDTAVYYCARMDGHGSPDYWGQGTLVTVSS

SEQ ID NO: 99 - protein, 124 aa, Artificial Sequence
  UNSURE (1)..(124): VH
QVQLLESGGGLVQPGGSLRLSCSASGFTFSSRAMHWVRQAPGKGLEYVSAISSNGGSTYYADSVKGRFTISRDNSKNTLYLQMSSLRAEDTAVYYCVIEGGSTGSTTSGAFDIWGQGTMVTVSS

SEQ ID NO: 100 - protein, 124 aa, Artificial Sequence
  UNSURE (1)..(124): VH
QVQLLESGGGLVQPGGSLRLSCSASGFTFSSYAMHWVRQAPGKGLEYVSAISSNGGSTYYADSVKGRFTISRDNSKNTLYLQMSSLRAEDTAVYYCVIEGGSTGSTVSGAFDIWGQGTMVTVSS

SEQ ID NO: 101 - protein, 112 aa, Artificial Sequence
  UNSURE (1)..(112): VL
DIVMTQSPLSLPVTLGQPASISCRSSQSLVYSDGNTYLNWFQQRPGQSPRRLIYEVSNRDSGVPDRFSGSGSGTDFTLKISRVEAEDVGVYYCMQGTHWPPAFGQGTKVEIK

SEQ ID NO: 102 - protein, 112 aa, Artificial Sequence
  UNSURE (1)..(112): VL
DIVMTQSPLSLPVTLGQPASISCRSSQSLVYSDGNTYLNWFQQRPGQSPRRLIYGVSNRDSGVPDRFSGSGSGTDFTLKISRVEAEDVGVYYCMQGTHWPPAFGQGTKVEIK

SEQ ID NO: 103 - protein, 112 aa, Artificial Sequence
  UNSURE (1)..(112): VL
DIVMTQSPLSLPVTLGQPASISCRSSQSLVYKDGNTYLNWFQQRPGQSPRRLIYKVSNRDSGVPDRFSGSGSGTDFTLKISRVEAEDVGVYYCMQGTHWPPRFGQGTKVEIK